(19) United States
(12) Patent Application Publication
(10) Pub. No.: US 2024/0243975 A1
(43) Pub. Date: Jul. 18, 2024

(54) USE OF INVERTED TERMINAL REPEATS (ITRS) FROM ADENO-ASSOCIATED VIRUS SEROTYPES 8 AND RH.39 IN GENE THERAPY VECTORS

(71) Applicant: University of Massachusetts, Boston, MA (US)

(72) Inventors: Guangping Gao, Worcester, MA (US); Phillip Tai, Worcester, MA (US); Elisabet C. Mandon, Worcester, MA (US); Jianzhong Ai, Worcester, MA (US)

(73) Assignee: University of Massachusetts, Boston, MA (US)

(21) Appl. No.: 18/432,467

(22) Filed: Feb. 5, 2024

Related U.S. Application Data
(63) Continuation of application No. 17/590,377, filed on Feb. 1, 2022.
(60) Provisional application No. 63/144,874, filed on Feb. 2, 2021.

(51) Int. Cl. H04L 41/12; H04L 41/22

(52) U.S. Cl. CPC H04L 41/12; H04L 41/22

(57) ABSTRACT

In some aspects, the disclosure provides recombinant AAV and nucleic acid constructs having novel inverted terminal repeats (ITRs), cap, and/or rep genes. In some aspects, the disclosure relates to gene transfer methods using rAAVs described herein.

### RELATED APPLICATIONS

This application claims the benefit under 35 U.S.C. 119(e) of the filing date of U.S. provisional application Ser. No. 63/144,874, filed Feb. 2, 2021, the entire contents of which are incorporated herein by reference.

### FEDERALLY SPONSORED RESEARCH

This invention was made with government support under NS076991, HL131471, HD080642, AI121135, HL147367, and W81XWH-17-1-0212 awarded by the National Institutes of Health and the Department of Defense. The government has certain rights in the invention.

### BACKGROUND OF INVENTION

Recombinant adeno-associated viruses (rAAVs) are capable of driving stable and sustained transgene expression in target tissues without notable toxicity and host immunogenicity. Approximately 94% of the genomic information within an AAV is replaced by a transgene of interest during preparation and design of an rAAV vector. The remaining 6% of the AAV genome, which typically includes two inverted terminal repeat (ITR) sequences that flank the AAV genome, are beneficial for replication and packaging during AAV production; and in providing stability and persistence when the rAAV vector is delivered to a host cell or subject. To date, several ITRs from different AAV serotypes have been described. Nonetheless, there is an increasing need to identify additional ITRs to improve the process of production and manufacturing, and the efficacy of AAV-based gene therapy.

### SUMMARY OF INVENTION

Aspects of the disclosure relate to isolated nucleic acid sequences encoding novel adeno-associated virus (AAV) components. The disclosure is based, in part, on novel configurations of AAV inverted terminal repeat (ITR) sequences and rAAV vectors comprising the same. In some embodiments, rAAV vectors described herein are surprisingly packaged into recombinant AAV (rAAV) particles, for example in a mammalian cell or insect cell production system. In some embodiments, rAAV vectors described herein are useful for delivering one or more transgenes to a cell or subject.

Accordingly, in some aspects, the disclosure provide a recombinant adeno-associated viral (rAAV) vector comprising a transgene flanked by inverted terminal repeats (ITRs), wherein a first ITR is an AAV8 ITR and a second ITR is an AAVrh.39 ITR.

In some embodiments, the first ITR is an AAV8 5′ ITR and the second ITR is an AAVrh.39 3′ ITR. In some embodiments, the first ITR comprises a nucleic acid sequence of SEQ ID NO: 2. In some embodiments, the second ITR comprises a nucleic acid sequence of SEQ ID NO: 3.

In some embodiments, the first ITR is an AAV8 3′ ITR and the second ITR is an AAVrh.39 5′ ITR. In some embodiments, the first ITR comprises a nucleic acid sequence of SEQ ID NO: 1. In some embodiments, the second ITR comprises a nucleic acid sequence of SEQ ID NO: 4.

In some aspects, the disclosure provides a recombinant adeno-associated viral (rAAV) vector comprising a transgene flanked by inverted terminal repeats (ITRs), wherein a first ITR comprises SEQ ID NO: 1. In some embodiments, the rAAV vector comprises a second ITR that comprises SEQ ID NO: 2.

In some aspects, the disclosure provides a recombinant adeno-associated viral (rAAV) vector comprising a transgene flanked by inverted terminal repeats (ITRs), wherein a first ITR comprises SEQ ID NO: 2. In some embodiments, the rAAV comprises a second ITR comprising SEQ ID NO: 1.

In some aspects, the disclosure provides a recombinant adeno-associated viral (rAAV) vector comprising a transgene flanked by inverted terminal repeats (ITRs), wherein a first ITR comprises SEQ ID NO: 3. In some embodiments, the rAAV vector comprises a second ITR that comprises SEQ ID NO: 4.

In some aspects, the disclosure provides a recombinant adeno-associated viral (rAAV) vector comprising a transgene flanked by inverted terminal repeats (ITRs), wherein a first ITR comprises SEQ ID NO: 3. In some embodiments, the rAAV vector comprises a second ITR that comprises SEQ ID NO: 4.

In some embodiments, a transgene encodes a protein or an inhibitory nucleic acid. In some embodiments, the inhibitory nucleic acid is a dsRNA, siRNA, shRNA, miRNA, artificial miRNA (amiRNA), or antisense oligonucleotide.

In some embodiments, an rAAV vector comprises a promoter operably linked to the transgene. In some embodiments, the promoter is a tissue specific promoter, inducible promoter, or constitutive promoter. In some embodiments, a tissue specific promoter is a liver-specific thyroxin binding globulin (TBG) promoter, an insulin promoter, a glucagon promoter, a somatostatin promoter, mucin-2 promoter, a pancreatic polypeptide (PPY) promoter, a synapsin-1 (Syn) promoter, a retinoschisin promoter, a K12 promoter, a CC10 promoter, a surfactant protein C (SP-C) promoter, a PRC1 promoter, a RRM2 promoter, uroplakin 2 (UPII) promoter, or a lactoferrin promoter. In some embodiments, a promoter is a cytomegalovirus (CMV) promoter, a CMV early enhancer/chicken β actin (CB6) promoter, or a T7 promoter.

In some aspects, the disclosure provides a host cell comprising an rAAV vector as described herein. In some embodiments, a host cell further comprises a Rep protein having the amino acid sequence set forth in any one of SEQ ID NOs: 7-14. In some embodiments, a host cell comprises a Cap protein having the amino acid sequence of SEQ ID NO: 15 or 16. In some embodiments, the host cell is a HEK293 cell or is derived from a HEK293 cell.

In some embodiments, the disclosure provides a nucleic acid comprising two inverted terminal repeats (ITRs), a rep gene, and a cap gene, wherein the rep gene encodes the amino acid sequence set forth in any one of SEQ ID NOs: 7-14. In some embodiments, the cap gene encodes the amino acid sequence set forth in SEQ ID NO: 15 or 16.

In some aspects, the disclosure provides a pseudotyped AAV comprising (i) an AAV8 3′ inverted terminal repeat (ITR) comprising SEQ ID NO: 1; and (ii) a capsid protein of a non-AAV8 AAV serotype.

In some embodiments, the pseudotyped AAV further comprises an AAV8 5′ ITR comprising SEQ ID NO: 2. In some embodiments, the pseudotyped AAV further comprises an AAVrh.39 5′ ITR comprising SEQ ID NO: 4.

In some embodiments, the disclosure provides a pseudotyped AAV comprising (i) an AAV8 5′ inverted terminal repeat (ITR) comprising SEQ ID NO: 2; and (ii) a capsid protein of a non-AAV8 AAV serotype. In some embodiments, the pseudotyped AAV further comprises an AAV8 3′ ITR comprising SEQ ID NO: 1. In some embodiments, the pseudotyped AAV further comprises an AAVrh.39 3′ ITR comprising SEQ ID NO: 3.

In some embodiments, the disclosure provides a pseudotyped AAV comprising (i) an AAVrh.39 3′ inverted terminal repeat (ITR) comprising SEQ ID NO: 3; and (ii) a capsid protein of a non-AAVrh.39 AAV serotype. In some embodiments, the pseudotyped AAV further comprises an AAVrh.39 5′ ITR comprising SEQ ID NO: 4. In some embodiments, the pseudotyped AAV further comprises an AAV8 5′ ITR comprising SEQ ID NO: 2.

In some aspects, the disclosure provides a pseudotyped AAV comprising (i) an AAVrh.39 5′ inverted terminal repeat (ITR) comprising SEQ ID NO: 4; and (ii) a capsid protein of a non-AAVrh.39 AAV serotype. In some embodiments, the pseudotyped AAV further comprises an AAVrh.39 3′ ITR comprising SEQ ID NO: 3. In some embodiments, the pseudotyped AAV further comprises an AAV8 3′ ITR comprising SEQ ID NO: 1.

In some embodiments, a pseudotyped AAV comprises a non-AAV8 AAV serotype selected from AAV1, AAV2, AAV3B, AAV9, AAVrh.8, AAVrh.10, and AAVrh.39. In some embodiments, a pseudotyped AAV comprises a non-AAVrh.39 AAV serotype selected from AAV1, AAV2, AAV3B, AAV8, AAV9, AAVrh.8, and AAVrh. 10.

In some embodiments, a pseudotyped AAV further comprises a transgene.

In some aspects, the disclosure provides a recombinant adeno-associated viral (rAAV) vector comprising a transgene flanked by inverted terminal repeats (ITRs), wherein a first ITR is an AAV2 ITR and a second ITR is an AAVrh.39 ITR or an AAV8 ITR.

In some embodiments, the first ITR comprises the nucleic acid sequence set forth in SEQ ID NO: 17. In some embodiments, the second ITR comprises the nucleic acid sequence set forth in any one of SEQ ID NOs: 1-4.

In some embodiments, the transgene encodes a therapeutic protein, one or more inhibitory nucleic acids, or a combination thereof.

In some embodiments, an rAAV vector further comprises one or more miRNA binding sites.

In some embodiments, the transgene comprises a promoter operably linked to a nucleic acid sequence encoding the therapeutic protein.

In some aspects, the disclosure provides a recombinant adeno-associated virus (rAAV) comprising: an rAAV vector of the disclosure; and an AAV capsid protein, optionally wherein the AAV capsid protein is not of the same serotype as any of the ITRs of the rAAV.

In some aspects, the disclosure provides a recombinant adeno-associated virus (rAAV) comprising (i) an AAV8 3′ inverted terminal repeat (ITR) comprising SEQ ID NO: 1; (ii) an AAVrh.39 5′ ITR comprising SEQ ID NO: 4 and (ii) an AAV8 capsid protein.

In some aspects, the disclosure provides a recombinant adeno-associated virus (rAAV) comprising (i) an AAV8 3′ inverted terminal repeat (ITR) comprising SEQ ID NO: 1; (ii) an AAVrh.39 5′ ITR comprising SEQ ID NO: 4 and (ii) an AAVrh.39 capsid protein.

In some aspects, the disclosure provides a recombinant adeno-associated virus (rAAV) comprising (i) an AAV8 5′ inverted terminal repeat (ITR) comprising SEQ ID NO: 2; (ii) an AAVrh.39 3′ ITR comprising SEQ ID NO: 3; and (iii) an AAV8 capsid protein.

In some aspects, the disclosure provides a recombinant adeno-associated virus (rAAV) comprising (i) an AAV8 5′ inverted terminal repeat (ITR) comprising SEQ ID NO: 2; (ii) an AAVrh.39 3′ ITR comprising SEQ ID NO: 3; and (iii) an AAVrh.39 capsid protein.

In some aspects, the disclosure provides a method for delivering a transgene to a cell or subject, the method comprising administering to the cell or subject an rAAV vector of the disclosure or an rAAV of the disclosure.

### BRIEF DESCRIPTION OF DRAWINGS

FIGS. 1A-1C depict embodiments of rAAV vector constructs and sequences of the disclosure. FIG. 1A shows a depiction of an rAAV vector genome comprising a transgene operably linked to a promoter and flanked by a 5′ ITR and a 3′ ITR. FIG. 1B shows a depiction of the sequence and structure of the AAV8 3′ ITR (SEQ ID NO: 1) of the disclosure. Rep binding elements (RBE) and a terminal resolution site (TRS) are highlighted. FIG. 1C shows a depiction of the sequence and structure of the AAVrh.39 3′ ITR (SEQ ID NO: 3) of the disclosure. Rep binding elements (RBE) and a terminal resolution site (TRS) are highlighted.

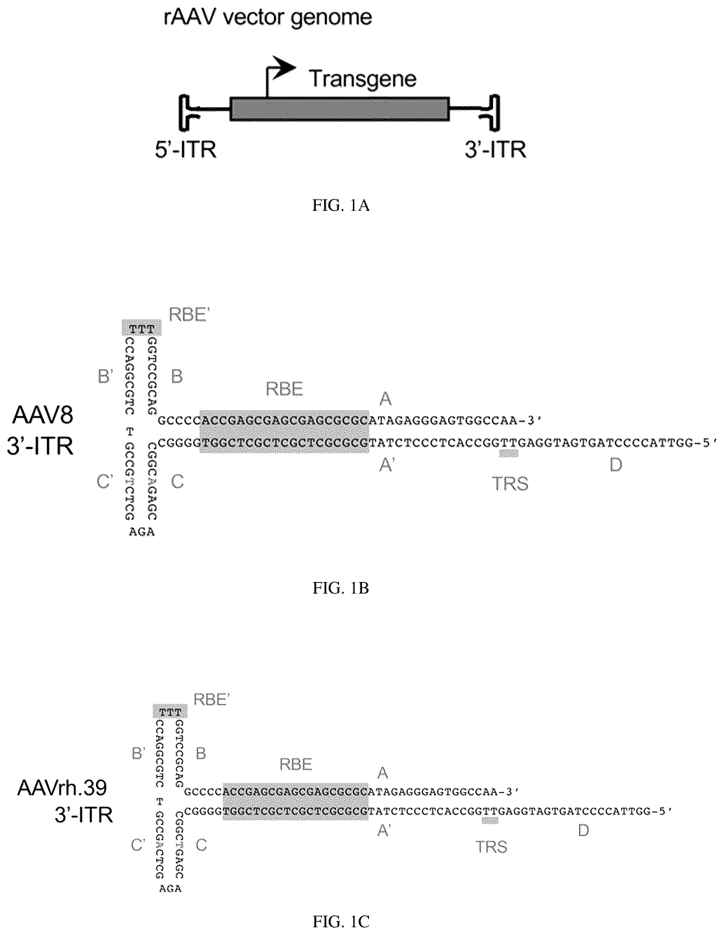

FIG. 2 depicts images of liver tissue from mice treated with rAAVs comprising hybrid ITR sequences of the disclosure.

FIGS. 3A-3C depict graphs showing the ability of rAAVs comprising hybrid ITR sequences of the disclosure to provide in vivo transgene expression. FIG. 3A provides a graph showing protein expression of eGFP in liver of mice treated with rAA Vs comprising hybrid ITR sequences of the disclosure. FIG. 3B provides a graph showing vector copies of rAAV in liver of mice treated with rAAVs comprising hybrid ITR sequences of the disclosure. FIG. 3C provides graphs showing gene expression of eGFP in liver of mice treated with rAAVs comprising hybrid ITR sequences of the disclosure.

FIGS. 4A-4B depict graphs showing the impact of varying ITR sequences at the 5′ end of a transgene and the 3′ end of a transgene.

FIG. 5 depicts graphs showing the ability of rAAVs comprising hybrid ITR sequences of the disclosure to provide stable in vivo transgene expression for up to three weeks in serum of mice treated with rAAVs comprising hybrid ITR sequences of the disclosure.

### DETAILED DESCRIPTION OF INVENTION

Gene delivery in laboratory and clinical settings has been achieved using vectors derived from adeno-associated viruses (AAVs). Current recombinant AAV (rAAV) vectors have approximately 94% of the AAV genome replaced with a expression cassette comprising a transgene. The remaining 6% of the viral genome is generally required for replication and packaging during production; and stability and persistence when the vector is delivered to the host cell. These viral components include one or two inverted terminal repeat (ITR) sequences that are generally 145-nucleotides in length, that flank the ends of the vector genome. The ITR structure forms a T-shaped hairpin. Engineering of the ITR structure through the introduction of mutations have enhanced the outcomes of the different steps in the viral life cycle. For example, changes at the terminal resolution site (TRS), which is required for viral replication, have been used to generate self-complementary AAV vectors (scAAVs). In the clinical context, importantly, ITR(s) are the only viral elements that remain in gene therapy vectors. These ITR sequences are directly linked with AAV genome integration and episomal persistence by circularization and concatemer formation of the vector genome following in vivo delivery. It is also thought that ITRs interact with host proteins and trigger anti-viral and DNA damage-response pathways. The ITRs are beneficial for the stability of AAV vectors, in part because they determine post-entry processing and persistence of the vector. Different ITR sequences (e.g., including the ITRs of the disclosure, e.g., SEQ ID NOs: 1-4) and/or different combinations of ITR sequences display distinctive patterns of vector genome metabolization, which indicates that varying ITR sequences can be manipulated to modulate vector stability, persistence, and safety.

Described herein are novel inverted terminal repeat (ITR) sequences identified as belonging to AAV8 and AAVrh.39 serotypes and the application of said ITR sequences in AAV compositions and vectors. In some embodiments, the AAV8 ITR sequence is a 3′ ITR sequence having a nucleic acid sequence as set forth in SEQ ID NO: 1. In some embodiments, the AAV8 ITR sequence is a 5′ ITR sequence having a nucleic acid sequence as set forth in SEQ ID NO: 2. In some embodiments, the AAVrh.39 ITR sequence is a 3′ ITR sequence having a nucleic acid sequence as set forth in SEQ ID NO: 3. In some embodiments, the AAVrh.39 ITR sequence is a 5′ ITR sequence having a nucleic acid sequence as set forth in SEQ ID NO: 4. In some embodiments, the AAV8 ITR sequences are an element of an AAV8 genome (e.g., an AAV8 genome having a nucleic acid sequence as set forth in SEQ ID NO: 5). In some embodiments, the AAVrh.39 ITR sequences are an element of an AAVrh.39 genome (e.g., an AAVrh.39 genome having a nucleic acid sequence as set forth in SEQ ID NO: 6).

In some embodiments, an AAV ITR of the disclosure is about 70%, about 75%, about 80%, about 85%, about 90%, about 95%, or about 99% identical to an ITR having a nucleic acid sequence of any one of SEQ ID NOs: 1-4. In some embodiments, an ITR is about 70%, about 75%, about 80%, about 85%, about 90%, about 95%, or about 99% identical to an ITR having a nucleic acid sequence of SEQ ID NO: 1. In some embodiments, an ITR is about 70%, about 75%, about 80%, about 85%, about 90%, about 95%, or about 99% identical to an ITR having a nucleic acid sequence of SEQ ID NO: 2. In some embodiments, an ITR is about 70%, about 75%, about 80%, about 85%, about 90%, about 95%, or about 99% identical to an ITR having a nucleic acid sequence of SEQ ID NO: 3. In some embodiments, an ITR is about 70%, about 75%, about 80%, about 85%, about 90%, about 95%, or about 99% identical to an ITR having a nucleic acid sequence of SEQ ID NO: 4.

Except for a small number of unpaired bases (e.g., 7 unpaired bases), an ITR sequence is self-complementary and forms an energetically stable “T” shaped hairpin (ΔG≈−72.4 kcal per mol, Tm>80° C.). An AAV ITR can exist in two conformations: “flip” and “flop”, which are a result of the rolling hairpin mechanism of AAV replication. During virus DNA replication, the cellular DNA polymerase complex initiates synthesis at the 3′-terminus of the template strand where the partial duplex formed by the ITR serves as the primer for primer extension. The replicative intermediate formed is an intramolecular duplex with the template and nascent strands covalently connected by the ITR. During a productive infection, a process called terminal resolution resolves the intra-strand ITR resulting in two full-length, complementary virus genomes.

In some embodiments, novel ITRs (e.g., AAV8 or AAVrh.39 ITRs, e.g., any one of SEQ ID NOs: 1-4) can be used to develop new “hybrid” ITR constructs to improve vector production, manufacturing, and in vivo performance. AAV ITRs interact with a number of host proteins and can trigger anti-viral and DNA damage response pathways. AAV vectors based on new hybrid ITR constructs have the potential to dampen host anti-viral and DNA damage-responses.

For example, a hybrid ITR construct may comprise an AAV8 ITR (e.g., a AAV8 5′ ITR or an AAV8 3′ ITR) and a non-AAV8 ITR. In some embodiments, a hybrid ITR construct comprises an AAV8 ITR and an AAVrh.39 ITR. In some embodiments, a hybrid ITR construct comprises an AAV8 5′ ITR and an AAVrh.39 3′ ITR. In some embodiments, a hybrid ITR construct comprises an AAV8 3′ ITR and an AAVrh.39 5′ ITR. In some embodiments, a hybrid ITR construct comprises an AAV8 ITR and an AAV2 ITR. In some embodiments, a hybrid ITR construct comprises an AAV8 5′ ITR and a 3′ AAV2 ITR. In some embodiments, a hybrid ITR construct comprises an AAV8 3′ ITR and an AAV2 5′ ITR.

In some embodiments, a hybrid ITR construct comprises an AAVrh.39 ITR (e.g., an AAVrh.39 5′ ITR or an AAVrh.39 3′ ITR) and a non-AAVrh.39 ITR. In some embodiments, a hybrid ITR construct comprises an AAVrh.39 ITR and an AAV2 ITR. In some embodiments, a hybrid ITR construct comprises an AAVrh.39 5′ ITR and a 3′ AAV2 ITR. In some embodiments, a hybrid ITR construct comprises an AAVrh.39 3′ ITR and an AAV2 5′ ITR.

In some embodiments, an AAV inverted terminal repeat sequence (e.g., an AAV8 ITR or and AAVrh.39 ITR) is an interrupted self-complementary sequence. Aspects of the disclosure include interrupted self-complementary nucleic acid sequences comprising an operative terminal resolution site (trs) that are useful for the formation of closed-ended linear duplex DNA (ccDNA). Typically, replication of nucleic acids comprising interrupted self-complementary nucleic acid sequences (e.g., AAV ITRs, e.g., comprising any one of SEQ ID NOs: 1-4) is initiated from the 3′ end of the cross-arm (e.g., hairpin structure) and generates a duplex molecule in which one of the ends is covalently closed; the covalently closed ends of the duplex molecule are then cleaved by a process called terminal resolution to form a two separate single-stranded nucleic acid molecules. Without wishing to be bound by any particular theory, the process of terminal resolution is mediated by a site- and strand-specific endonuclease cleavage at a terminal resolution site (trs) (e.g., a rolling circle replication protein, such as AAV Rep protein). Examples of trs sequences include 3′-CCGGTTG-5 and 5′-AGTTGG-3′. It has been hypothesized that Rep-mediated strand nicking takes place between the central di-thymidine (“TT”) portion of the trs sequence. Therefore, in some embodiments, the operative terminal resolution site comprises a sequence 5′-TT-3′.

Aspects of the disclosure relate to the positioning of a terminal resolution site (trs) relative to a rolling circle replication protein binding element. Generally, a trs is positioned upstream (e.g., 5′) relative to a rolling circle replication protein binding element. However, in some embodiments, a trs is positioned downstream (e.g., 3′) relative to a rolling circle replication protein binding element. In some embodiments, the 3′ end of the operative terminal resolution site is 15 to 25 nucleotides (e.g., 15, 16, 17, 18, 19, 20, 21, 22, 23, 24, or 25 nucleotides) from the 5′ end of the rolling circle replication protein binding element.

In the absence of AAV cap gene expression, an AAV vector genome having asymmetrical ITRs undergoes inefficient replication, and the replication products accumulate in a novel conformation of closed-ended linear duplex DNA (ceDNA). Due to inefficient replication (e.g., incomplete terminal resolution), the complementary strands of the intramolecular intermediate are now covalently linked through the ITRs on both ends of the genome. Thus, in native conditions, ceDNA behaves as linear duplex DNA, however, in denaturing conditions, ceDNA strands melt apart, but remain linked, therefore transforming the linear duplex molecule into single-stranded circular DNA.

In some embodiments, ceDNA production from asymmetric AAV ITRs is dependent on a truncated ITR at one end and an operative (e.g., functional) ITR (e.g., an ITRs comprising any one of SEQ ID NOs: 1-4) on the opposite end of the transgene cassette. In some embodiments, the truncated ITR is inefficiently processed during the replication cycles of ceDNA in host cells leading to an accumulation of replication intermediates, duplex monomer, duplex dimers, etc. In the absence of structural (capsid or cap) protein expression, a Rep protein (e.g., Rep 78 or Rep 68) assembles on the intact ITRs and catalyze the site-specific nicking at the terminal resolution site. The reaction results in the formation of a transient tyrosine-phosphodiester between a tyrosine of the Rep protein and a thymidine of the terminal resolution site of the ITS. The transient nucleoprotein complex then transfers the donor strand to the free 3′-terminus of the complementary strand. The ceDNA conformation therefore, results from the defective ITR on one end of the vector genome, an intact or operative ITR on the opposite end, and the co-expression of the Rep proteins (e.g., p5 and p19 Rep proteins, where at least one p5 and one p19 Rep are expressed). Other parvovirus “small” Rep or NS proteins can substitute for AAV p19 Rep protein (Rep 52 and Rep 40). Since these small Rep proteins are non-processive, monomeric helicases, it is feasible that non-parvoviridae super family 2 (SF2) helicases can substitute for the AAV Rep proteins.

In some embodiments, a Rep protein is a Rep78, Rep68, Rep52, or Rep 40 protein. In some embodiments, an AAV8 Rep78 protein comprises the amino acid sequence as set forth in SEQ ID NO: 7. In some embodiments, an AAV8 Rep68 protein comprises the amino acid sequence as set forth in SEQ ID NO: 8. In some embodiments, an AAV8 Rep52 protein comprises the amino acid sequence as set forth in SEQ ID NO: 9. In some embodiments, an AAV8 Rep40 protein comprises the amino acid sequence as set forth in SEQ ID NO: 10. In some embodiments, an AAVrh.39 Rep78 protein comprises the amino acid sequence as set forth in SEQ ID NO: 11. In some embodiments, an AAVrh.39 Rep68 protein comprises the amino acid sequence the set forth in SEQ ID NO: 12. In some embodiments, an AAVrh.39 Rep52 protein comprises the amino acid sequence as set forth in SEQ ID NO: 13. In some embodiments, an AAVrh.39 Rep40 protein comprises the amino acid sequence as set forth in SEQ ID NO: 14.

In some embodiments, a cap protein is an AAV8 cap protein. In some embodiments, an AAV8 cap protein comprises the amino acid sequence as set forth in SEQ ID NO: 15. In some embodiments, a cap protein is an AAVrh.39 cap protein. In some embodiments, an AAVrh.39 cap protein comprises the amino acid sequence as set forth in SEQ ID NO: 16.

Recombinant AAV (rAAV) vectors of the disclosure are typically composed of, at a minimum, a transgene and its regulatory sequences, and 5′ and 3′ AAV ITRs (e.g., AAV ITRs comprising any one of SEQ ID NOs: 1-4). In some embodiments, a recombinant AAV vector is packaged into a capsid protein and delivered to a selected target cell. In some embodiments, the transgene is a nucleic acid sequence, heterologous to the vector sequences, that encodes a polypeptide, protein, functional RNA molecule (e.g., miRNA, miRNA inhibitor) or other gene product, of interest. The nucleic acid coding sequence is operatively linked to regulatory components in a manner that permits transgene transcription, translation, and/or expression in a cell of a target tissue.

The AAV sequences of the vector comprise the cis-acting 5′ and 3′ inverted terminal repeat sequences (e.g., comprising any one of SEQ ID NOs: 1-4). In some embodiments, the ITR sequences are about 145 bp in length. Preferably, substantially the entire sequences encoding the ITRs are used in the molecule, although some degree of minor modification of these sequences is permissible. The ability to modify nucleic acid sequences is within the skill of the art. (Sec, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of such a molecule employed in the present disclosure is a “cis-acting” plasmid containing the transgene, in which the selected transgene sequence and associated regulatory elements are flanked by the 5′ and 3′ AAV ITR sequences.

As used herein, the term “flanked” refers to the positioning of a first interrupted self-complementary sequence upstream (e.g., 5′) relative to a heterologous nucleic acid insert and a second interrupted self-complementary sequence downstream (e.g., 3′) relative to the heterologous nucleic acid insert. For example, an adeno-associated virus genome comprises open reading frames of the rep and cap genes “flanked” by inverted terminal repeats (ITRs).

As used herein, the term “operative” refers to the ability of a nucleic acid sequence to perform its intended function. For example, an “operative binding region” is a nucleic acid sequence that retains binding function for its intended target (e.g., a protein or nucleic acid). In another example, an “operative cleavage site” is a nucleic acid sequence that retains its ability to be specifically cleaved by a particular enzyme or enzymes.

In some embodiments, the disclosure provides a self-complementary AAV vector. As used herein, the term “self-complementary AAV vector” (scAAV) refers to a vector containing a double-stranded vector genome generated by the absence of a terminal resolution site (TR) from one of the ITRs (e.g., an ITR comprising any one of SEQ ID NOs: 1-4). The absence of a TR prevents the initiation of replication at the vector terminus where the TR is not present. In general, scAAV vectors generate single-stranded, inverted repeat genomes, with a wild-type (wt) AAV TR at each end and a mutated TR (mTR) in the middle.

In some embodiments, the rAAVs of the present disclosure are pseudotyped rAAVs. Pseudotyping is the process of producing viruses or viral vectors in combination with foreign viral envelope proteins. The result is a pseudotyped virus particle. With this method, the foreign viral envelope proteins can be used to alter host tropism or an increased/decreased stability of the virus particles. In some aspects, a pseudotyped rAAV comprises nucleic acids from two or more different AAVs, wherein the nucleic acid from one AAV encodes a capsid protein and the nucleic acid of at least one other AAV encodes other viral proteins and/or the viral genome. In some embodiments, a pseudotyped rAAV refers to an AAV comprising an inverted terminal repeat (ITR) of one AAV serotype and a capsid protein of a different AAV serotype. For example, a pseudotyped AAV vector containing the ITRs of serotype X encapsidated with the proteins of Y will be designated as AAVX/Y (e.g., AAV2/1 has the ITRs of AAV2 and the capsid of AAV1). In some embodiments, pseudotyped rAAVs may be useful for combining the tissue-specific targeting capabilities of a capsid protein from one AAV serotype with the viral DNA from another AAV serotype, thereby allowing targeted delivery of a transgene to a target tissue.

In some embodiments, the rAAVs of the present disclosure comprise at least one AAV8 ITR sequence and an AAV8 capsid protein. In some embodiments, an rAAV comprises two AAV8 ITR sequences (an AAV8 5′ ITR and an AAV8 3′ ITR) and an AAV8 capsid protein. In some embodiments, an rAAV comprises an AAV8 ITR sequence, an AAVrh.39 ITR sequence, and an AAV8 capsid protein. In some embodiments, an rAAV comprises an AAV8 5′ ITR sequence, an AAVrh.39 3′ ITR sequence, and an AAV8 capsid protein. In some embodiments, an rAAV comprises an AAV8 3′ ITR sequence, an AAVrh.39 5′ ITR sequence, and an AAV8 capsid protein.

In some embodiments, the rAAVs of the present disclosure comprise at least one AAVrh.39 ITR sequence and an AAVrh.39 capsid protein. In some embodiments, an rAAV comprises two AAVrh.39 ITR sequences (an AAVrh.39 5′ ITR and an AAVrh.39 3′ ITR) and an AAVrh.39 capsid protein. In some embodiments, an rAAV comprises an AAV8 ITR sequence, an AAVrh.39 ITR sequence, and an AAVrh.39 capsid protein. In some embodiments, an rAAV comprises an AAV8 5′ ITR sequence, an AAVrh.39 3′ ITR sequence, and an AAVrh.39 capsid protein. In some embodiments, an rAAV comprises an AAV8 3′ ITR sequence, an AAVrh.39 5′ ITR sequence, and an AAVrh.39 capsid protein.

As used here, “rolling circle replication protein binding element” refers to a conserved nucleic acid sequence (e.g., motif) that is recognized and bound by a rolling circle replication protein, which is a viral nonstructural protein (NS protein) that initiates rolling circle (e.g., rolling hairpin) replication. Rolling circle (e.g., rolling hairpin) replication is described by Tattersall et al. Nature 2009, 263, pp. 106-109. Examples of NS proteins include, but are not limited to AAV Rep proteins (e.g., Rep78, Rep68, Rep52, Rep40), parvovirus nonstructural proteins (e.g., NS2), rotavirus nonstructural proteins (e.g., NSP1), and densovirus nonstructural proteins (e.g., PfDNV NS1). In some embodiments, the rolling circle replication protein binding element is a Rep binding element (RBE). In some embodiments, the RBE comprises the sequence 5′-GCTCGCTCGCTC-3′ (SEQ ID NO: 18).

In some embodiments, rolling circle replication proteins are from the dependoparvovirus genus of the Parvoviridae family of viruses with linear single-stranded DNA genomes. In some embodiments, the rolling circle replication proteins are from the genera of the autonomous Parvovirinae including mice minute virus, Aleutian mink disease virus, bovine parvovirus, canine parvovirus, chicken parvovirus, feline panleukopenia virus, feline parvovirus, goose parvovirus, HB parvovirus, H-1 parvovirus, Kilham rat virus, lapine parvovivirus, LUIII virus, mink enteritis virus, mouse parvovirus, porcine parvovirus, raccoon parvovirus, RT parvovirus, Tumor virus X, rat parvovirus 1a, barbaric duck parvovirus, equine parvovirus, hamster parvovirus, and rheumatoid arthritis virus 1. In some embodiments, the genus is parvovirus. In some embodiments, the rolling circle replication proteins are from the genera of Densovirinae including brevidensovirus, densovirus, and iteravirus.

In some embodiments, a rolling circle replication protein is from the genera of the subfamily Parvovirinae. Examples of Parvovirinae genera include but are not limited to Amdoparvovirus, Aveparvovirus, Bocaparvovirus, Copiparvovirus, Dependoparvovirus, Erythroparvovirus, Protoparvovirus, and Tetraparvovirus. In some embodiments, the rolling circle replication proteins are from the genera of the subfamily, Densovirinae. Examples of Densovirinae genera include but are not limited to Amdoparvovirus, Aveparvovirus, Bocaparvovirus, Copiparvovirus, Dependoparvovirus, Erythroparvovirus, Protoparvovirus, and Tetraparvovirus. In some embodiments, the rolling circle replication protein(s) is from a Dependovirus, such as Adeno-associated virus 2 (AAV2), Adeno-associated virus 3 (AAV3), Adeno-associated virus 4 (AAV4), or Adeno-associated virus 5 (AAV5), or any combination thereof.

In some embodiments, the rolling circle replication proteins are derived from the single-stranded DNA bacteriophage families. In some embodiments, the virus families are the Microviridae and the Inoviridae. In some embodiments, the rolling circle replication proteins are derived from Gram positive bacteria.

In addition to the major elements identified above for the nucleic acid, the nucleic acid also includes conventional control elements necessary which are operably linked to the heterologous nucleic acid insert (e.g., transgene) in a manner which permits its transcription, translation and/or expression in a cell transfected with a nucleic acid described by the disclosure. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

In some embodiments, a heterologous nucleic acid insert (e.g., transgene) comprises a protein coding sequence that is operably linked to one or more regulatory sequences. As used herein, a nucleic acid coding sequence and regulatory sequences are said to be “operably” linked when they are covalently linked in such a way as to place the expression or transcription of the nucleic acid sequence (e.g., transgene) under the influence or control of the regulatory sequences. If it is desired that the nucleic acid sequence (e.g., transgene) be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., gRNA).

In some embodiments, proteins and nucleic acids of the disclosure are isolated. As used herein, the term “isolated” means artificially produced. In some embodiments, with respect to nucleic acids, the term “isolated” refers to a nucleic acid that is: (i) amplified in vitro by, for example, polymerase chain reaction (PCR); (ii) recombinantly produced by molecular cloning; (iii) purified, as by restriction endonuclease cleavage and gel electrophoretic fractionation, or column chromatography; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

For nucleic acids (e.g., transgenes) encoding proteins, a polyadenylation sequence generally is inserted following the transgene sequences and before the 3′ AAV ITR sequence. A heterologous nucleic acid insert (e.g., transgene) useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al, and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931.; and Klump, H et al., Gene Therapy, 2001; 8: 811-817).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the disclosure may optionally include 5′ leader or signal sequences. The choice and design of an appropriate vector is within the ability and discretion of one of ordinary skill in the art.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al, Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

The composition of the transgene sequence (e.g., heterologous nucleic acid insert) of the nucleic acid will depend upon the use to which the resulting nucleic acid will be put. For example, one type of transgene sequence includes a reporter sequence, which upon expression produces a detectable signal. In another example, the transgene encodes a therapeutic protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

In some embodiments, a transgene (e.g., heterologous nucleic acid insert) flanked by ITRs ranges from about 10 to about 5,000 base pairs, about 10 to about 10,000 base pairs, about 10 to about 50,000 base pairs in length. In some embodiments, a transgene (e.g., heterologous nucleic acid insert) flanked by ITRs ranges from about 10 to about 50 base pairs in length. In some embodiments, a transgene (e.g., heterologous nucleic acid insert) flanked by ITRs ranges from about 20 to about 100 base pairs in length. In some embodiments, a transgene (e.g., heterologous nucleic acid insert) flanked by ITRs ranges from about 500 to about 1500 base pairs in length. In some embodiments, a transgene (e.g., heterologous nucleic acid insert) flanked by ITRs ranges from about 1000 to about 5000 base pairs in length. In some embodiments, the size of a transgene (e.g., heterologous nucleic acid insert) exceeds the capacity of a traditional AAV vector (e.g., exceeds about 4.8 kb).

Reporter sequences that may be provided in a transgene include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer. Such reporters can, for example, be useful in verifying the tissue-specific targeting capabilities and tissue specific promoter regulatory activity of a nucleic acid.

In some aspects, the disclosure provides nucleic acids for use in methods of preventing or treating one or more genetic deficiencies or dysfunctions in a mammal, such as for example, a polypeptide deficiency or polypeptide excess in a mammal, and particularly for treating or reducing the severity or extent of deficiency in a human manifesting one or more of the disorders linked to a deficiency in such polypeptides in cells and tissues. The method involves administration of nucleic acid (e.g., a nucleic acid as described by the disclosure) that encodes one or more therapeutic peptides, polypeptides, siRNAs, microRNAs, antisense nucleotides, etc. in a pharmaceutically-acceptable carrier to the subject in an amount and for a period of time sufficient to treat the deficiency or disorder in the subject suffering from such a disorder.

Thus, the disclosure embraces the delivery of nucleic acids (e.g., nucleic acids as described by the disclosure) encoding one or more peptides, polypeptides, or proteins, which are useful for the treatment or prevention of disease states in a mammalian subject. Exemplary therapeutic proteins include one or more polypeptides selected from the group consisting of growth factors, interleukins, interferons, anti-apoptosis factors, cytokines, anti-diabetic factors, anti-apoptosis agents, coagulation factors, anti-tumor factors. Other non-limiting examples of therapeutic proteins include BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAP1, IL-1, IL-2, IL-3, IL-4, IL-5, IL-6, IL-7, IL-8, IL-9, IL-10, IL-10(187A), viral IL-10, IL-11, IL-12, IL-13, IL-14, IL-15, IL-16 IL-17, and IL-18.

The nucleic acids (e.g., nucleic acids as described by the disclosure) may comprise a gene to be transferred (e.g., expressed in) to a subject to treat a disease associated with reduced expression, lack of expression or dysfunction of the gene. Exemplary genes and associated disease states include, but are not limited to: glucose-6-phosphatase, associated with glycogen storage deficiency type 1A; phosphoenolpyruvate-carboxykinase, associated with Pepck deficiency; galactose-1 phosphate uridyl transferase, associated with galactosemia;

phenylalanine hydroxylase, associated with phenylketonuria; branched chain alpha-ketoacid dehydrogenase, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; omithine transcarbamylase, associated with omithine transcarbamylase deficiency; argininosuccinic acid synthetase, associated with citrullinemia; low density lipoprotein receptor protein, associated with familial hypercholesterolemia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotinidase, associated with biotinidase deficiency; beta-glucocerebrosidase, associated with Gaucher disease; beta-glucuronidase, associated with Sly syndrome; peroxisome membrane protein 70 kDa, associated with Zellweger syndrome; porphobilinogen deaminase, associated with acute intermittent porphyria; alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin for the treatment of diabetes.

The nucleic acids of the disclosure (e.g., nucleic acid having a heterologous nucleic acid insert) can be used to restore the expression of genes that are reduced in expression, silenced, or otherwise dysfunctional in a subject (e.g., a tumor suppressor that has been silenced in a subject having cancer). The nucleic acids of the disclosure can also be used to knockdown the expression of genes that are aberrantly expressed in a subject (e.g., an oncogene that is expressed in a subject having cancer). In some embodiments, a heterologous nucleic acid insert encoding a gene product associated with cancer (e.g., tumor suppressors) may be used to treat the cancer, by administering nucleic acid comprising the heterologous nucleic acid insert to a subject having the cancer. In some embodiments, a nucleic acid comprising a heterologous nucleic acid insert encoding a small interfering nucleic acid (e.g., shRNAs, miRNAs) that inhibits the expression of a gene product associated with cancer (e.g., oncogenes) may be used to treat the cancer, by administering nucleic acid comprising the heterologous nucleic acid insert to a subject having the cancer. In some embodiments, nucleic comprising a heterologous nucleic acid insert encoding a gene product associated with cancer (or a functional RNA that inhibits the expression of a gene associated with cancer) may be used for research purposes, e.g., to study the cancer or to identify therapeutics that treat the cancer.

The skilled artisan will also realize that in the case of transgenes encoding proteins or polypeptides, that mutations that results in conservative amino acid substitutions may be made in a transgene to provide functionally equivalent variants, or homologs of a protein or polypeptide. In some aspects the disclosure embraces sequence alterations that result in conservative amino acid substitution of a transgene. In some embodiments, the transgene comprises a gene having a dominant negative mutation. For example, a transgene may express a mutant protein that interacts with the same elements as a wild-type protein, and thereby blocks some aspect of the function of the wild-type protein.

Useful transgene products also include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

The following non-limiting list of miRNA genes, and their homologues, are useful as transgenes or as targets for small interfering nucleic acids encoded by transgenes (e.g., miRNA sponges, antisense oligonucleotides, TuD RNAs) in certain embodiments of the methods: hsa-let-7a, hsa-let-7a*, hsa-let-7b, hsa-let-7b*, hsa-let-7c, hsa-let-7c*, hsa-let-7d, hsa-let-7d*, hsa-let-7c, hsa-let-7c*, hsa-let-7f, hsa-let-7f-1*, hsa-let-7f-2*, hsa-let-7g, hsa-let-7g*, hsa-let-7i, hsa-let-7i*, hsa-miR-1, hsa-miR-100, hsa-miR-100*, hsa-miR-101, hsa-miR-101*, hsa-miR-103, hsa-miR-105, hsa-miR-105*, hsa-miR-106a, hsa-miR-106a*, hsa-miR-106b, hsa-miR-106b*, hsa-miR-107, hsa-miR-10a, hsa-miR-10a*, hsa-miR-10b, hsa-miR-10b*, hsa-miR-1178, hsa-miR-1179, hsa-miR-1180, hsa-miR-1181, hsa-miR-1182, hsa-miR-1183, hsa-miR-1184, hsa-miR-1185, hsa-miR-1197, hsa-miR-1200, hsa-miR-1201, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-122, hsa-miR-122*, hsa-miR-1224-3p, hsa-miR-1224-5p, hsa-miR-1225-3p, hsa-miR-1225-5p, hsa-miR-1226, hsa-miR-1226*, hsa-miR-1227, hsa-miR-1228, hsa-miR-1228*, hsa-miR-1229, hsa-miR-1231, hsa-miR-1233, hsa-miR-1234, hsa-miR-1236, hsa-miR-1237, hsa-miR-1238, hsa-miR-124, hsa-miR-124*, hsa-miR-1243, hsa-miR-1244, hsa-miR-1245, hsa-miR-1246, hsa-miR-1247, hsa-miR-1248, hsa-miR-1249, hsa-miR-1250, hsa-miR-1251, hsa-miR-1252, hsa-miR-1253, hsa-miR-1254, hsa-miR-1255a, hsa-miR-1255b, hsa-miR-1256, hsa-miR-1257, hsa-miR-1258, hsa-miR-1259, hsa-miR-125a-3p, hsa-miR-125a-5p, hsa-miR-125b, hsa-miR-125b-1*, hsa-miR-125b-2*, hsa-miR-126, hsa-miR-126*, hsa-miR-1260, hsa-miR-1261, hsa-miR-1262, hsa-miR-1263, hsa-miR-1264, hsa-miR-1265, hsa-miR-1266, hsa-miR-1267, hsa-miR-1268, hsa-miR-1269, hsa-miR-1270, hsa-miR-1271, hsa-miR-1272, hsa-miR-1273, hsa-miR-127-3p, hsa-miR-1274a, hsa-miR-1274b, hsa-miR-1275, hsa-miR-127-5p, hsa-miR-1276, hsa-miR-1277, hsa-miR-1278, hsa-miR-1279, hsa-miR-128, hsa-miR-1280, hsa-miR-1281, hsa-miR-1282, hsa-miR-1283, hsa-miR-1284, hsa-miR-1285, hsa-miR-1286, hsa-miR-1287, hsa-miR-1288, hsa-miR-1289, hsa-miR-129*, hsa-miR-1290, hsa-miR-1291, hsa-miR-1292, hsa-miR-1293, hsa-miR-129-3p, hsa-miR-1294, hsa-miR-1295, hsa-miR-129-5p, hsa-miR-1296, hsa-miR-1297, hsa-miR-1298, hsa-miR-1299, hsa-miR-1300, hsa-miR-1301, hsa-miR-1302, hsa-miR-1303, hsa-miR-1304, hsa-miR-1305, hsa-miR-1306, hsa-miR-1307, hsa-miR-1308, hsa-miR-130a, hsa-miR-130a*, hsa-miR-130b, hsa-miR-130b*, hsa-miR-132, hsa-miR-132*, hsa-miR-1321, hsa-miR-1322, hsa-miR-1323, hsa-miR-1324, hsa-miR-133a, hsa-miR-133b, hsa-miR-134, hsa-miR-135a, hsa-miR-135a*, hsa-miR-135b, hsa-miR-135b*, hsa-miR-136, hsa-miR-136*, hsa-miR-137, hsa-miR-138, hsa-miR-138-1*, hsa-miR-138-2*, hsa-miR-139-3p, hsa-miR-139-5p, hsa-miR-140-3p, hsa-miR-140-5p, hsa-miR-141, hsa-miR-141*, hsa-miR-142-3p, hsa-miR-142-5p, hsa-miR-143, hsa-miR-143*, hsa-miR-144, hsa-miR-144*, hsa-miR-145, hsa-miR-145*, hsa-miR-146a, hsa-miR-146a*, hsa-miR-146b-3p, hsa-miR-146b-5p, hsa-miR-147, hsa-miR-147b, hsa-miR-148a, hsa-miR-148a*, hsa-miR-148b, hsa-miR-148b*, hsa-miR-149, hsa-miR-149*, hsa-miR-150, hsa-miR-150*, hsa-miR-151-3p, hsa-miR-151-5p, hsa-miR-152, hsa-miR-153, hsa-miR-154, hsa-miR-154*, hsa-miR-155, hsa-miR-155*, hsa-miR-15a, hsa-miR-15a*, hsa-miR-15b, hsa-miR-15b*, hsa-miR-16, hsa-miR-16-1*, hsa-miR-16-2*, hsa-miR-17, hsa-miR-17*, hsa-miR-181a, hsa-miR-181a*, hsa-miR-181a-2*, hsa-miR-181b, hsa-miR-181c, hsa-miR-181c*, hsa-miR-181d, hsa-miR-182, hsa-miR-182*, hsa-miR-1825, hsa-miR-1826, hsa-miR-1827, hsa-miR-183, hsa-miR-183*, hsa-miR-184, hsa-miR-185, hsa-miR-185*, hsa-miR-186, hsa-miR-186*, hsa-miR-187, hsa-miR-187*, hsa-miR-188-3p, hsa-miR-188-5p, hsa-miR-18a, hsa-miR-18a*, hsa-miR-18b, hsa-miR-18b*, hsa-miR-190, hsa-miR-190b, hsa-miR-191, hsa-miR-191*, hsa-miR-192, hsa-miR-192*, hsa-miR-193a-3p, hsa-miR-193a-5p, hsa-miR-193b, hsa-miR-193b*, hsa-miR-194, hsa-miR-194*, hsa-miR-195, hsa-miR-195*, hsa-miR-196a, hsa-miR-196a*, hsa-miR-196b, hsa-miR-197, hsa-miR-198, hsa-miR-199a-3p, hsa-miR-199a-5p, hsa-miR-199b-5p, hsa-miR-19a, hsa-miR-19a*, hsa-miR-19b, hsa-miR-19b-1*, hsa-miR-19b-2*, hsa-miR-200a, hsa-miR-200a*, hsa-miR-200b, hsa-miR-200b*, hsa-miR-200c, hsa-miR-200c*, hsa-miR-202, hsa-miR-202*, hsa-miR-203, hsa-miR-204, hsa-miR-205, hsa-miR-206, hsa-miR-208a, hsa-miR-208b, hsa-miR-20a, hsa-miR-20a*, hsa-miR-20b, hsa-miR-20b*, hsa-miR-21, hsa-miR-21*, hsa-miR-210, hsa-miR-211, hsa-miR-212, hsa-miR-214, hsa-miR-214*, hsa-miR-215, hsa-miR-216a, hsa-miR-216b, hsa-miR-217, hsa-miR-218, hsa-miR-218-1*, hsa-miR-218-2*, hsa-miR-219-1-3p, hsa-miR-219-2-3p, hsa-miR-219-5p, hsa-miR-22, hsa-miR-22*, hsa-miR-220a, hsa-miR-220b, hsa-miR-220c, hsa-miR-221, hsa-miR-221*, hsa-miR-222, hsa-miR-222*, hsa-miR-223, hsa-miR-223*, hsa-miR-224, hsa-miR-23a, hsa-miR-23a*, hsa-miR-23b, hsa-miR-23b*, hsa-miR-24, hsa-miR-24-1*, hsa-miR-24-2*, hsa-miR-25, hsa-miR-25*, hsa-miR-26a, hsa-miR-26a-1*, hsa-miR-26a-2*, hsa-miR-26b, hsa-miR-26b*, hsa-miR-27a, hsa-miR-27a*, hsa-miR-27b, hsa-miR-27b*, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a, hsa-miR-29a*, hsa-miR-29b, hsa-miR-29b-1*, hsa-miR-29b-2*, hsa-miR-29c, hsa-miR-29c*, hsa-miR-300, hsa-miR-301a, hsa-miR-301b, hsa-miR-302a, hsa-miR-302a*, hsa-miR-302b, hsa-miR-302b*, hsa-miR-302c, hsa-miR-302c*, hsa-miR-302d, hsa-miR-302d*, hsa-miR-302c, hsa-miR-302f, hsa-miR-30a, hsa-miR-30a*, hsa-miR-30b, hsa-miR-30b*, hsa-miR-30c, hsa-miR-30c-1*, hsa-miR-30c-2*, hsa-miR-30d, hsa-miR-30d*, hsa-miR-30c, hsa-miR-30c*, hsa-miR-31, hsa-miR-31*, hsa-miR-32, hsa-miR-32*, hsa-miR-320a, hsa-miR-320b, hsa-miR-320c, hsa-miR-320d, hsa-miR-323-3p, hsa-miR-323-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335, hsa-miR-335*, hsa-miR-337-3p, hsa-miR-337-5p, hsa-miR-338-3p, hsa-miR-338-5p, hsa-miR-339-3p, hsa-miR-339-5p, hsa-miR-33a, hsa-miR-33a*, hsa-miR-33b, hsa-miR-33b*, hsa-miR-340, hsa-miR-340*, hsa-miR-342-3p, hsa-miR-342-5p, hsa-miR-345, hsa-miR-346, hsa-miR-34a, hsa-miR-34a*, hsa-miR-34b, hsa-miR-34b*, hsa-miR-34c-3p, hsa-miR-34c-5p, hsa-miR-361-3p, hsa-miR-361-5p, hsa-miR-362-3p, hsa-miR-362-5p, hsa-miR-363, hsa-miR-363*, hsa-miR-365, hsa-miR-367, hsa-miR-367*, hsa-miR-369-3p, hsa-miR-369-5p, hsa-miR-370, hsa-miR-371-3p, hsa-miR-371-5p, hsa-miR-372, hsa-miR-373, hsa-miR-373*, hsa-miR-374a, hsa-miR-374a*, hsa-miR-374b, hsa-miR-374b*, hsa-miR-375, hsa-miR-376a, hsa-miR-376a*, hsa-miR-376b, hsa-miR-376c, hsa-miR-377, hsa-miR-377*, hsa-miR-378, hsa-miR-378*, hsa-miR-379, hsa-miR-379*, hsa-miR-380, hsa-miR-380*, hsa-miR-381, hsa-miR-382, hsa-miR-383, hsa-miR-384, hsa-miR-409-3p, hsa-miR-409-5p, hsa-miR-410, hsa-miR-411, hsa-miR-411*, hsa-miR-412, hsa-miR-421, hsa-miR-422a, hsa-miR-423-3p, hsa-miR-423-5p, hsa-miR-424, hsa-miR-424*, hsa-miR-425, hsa-miR-425*, hsa-miR-429, hsa-miR-431, hsa-miR-431*, hsa-miR-432, hsa-miR-432*, hsa-miR-433, hsa-miR-448, hsa-miR-449a, hsa-miR-449b, hsa-miR-450a, hsa-miR-450b-3p, hsa-miR-450b-5p, hsa-miR-451, hsa-miR-452, hsa-miR-452*, hsa-miR-453, hsa-miR-454, hsa-miR-454*, hsa-miR-455-3p, hsa-miR-455-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a, hsa-miR-487b, hsa-miR-488, hsa-miR-488*, hsa-miR-489, hsa-miR-490-3p, hsa-miR-490-5p, hsa-miR-491-3p, hsa-miR-491-5p, hsa-miR-492, hsa-miR-493, hsa-miR-493*, hsa-miR-494, hsa-miR-495, hsa-miR-496, hsa-miR-497, hsa-miR-497*, hsa-miR-498, hsa-miR-499-3p, hsa-miR-499-5p, hsa-miR-500, hsa-miR-500*, hsa-miR-501-3p, hsa-miR-501-5p, hsa-miR-502-3p, hsa-miR-502-5p, hsa-miR-503, hsa-miR-504, hsa-miR-505, hsa-miR-505*, hsa-miR-506, hsa-miR-507, hsa-miR-508-3p, hsa-miR-508-5p, hsa-miR-509-3-5p, hsa-miR-509-3p, hsa-miR-509-5p, hsa-miR-510, hsa-miR-511, hsa-miR-512-3p, hsa-miR-512-5p, hsa-miR-513a-3p, hsa-miR-513a-5p, hsa-miR-513b, hsa-miR-513c, hsa-miR-514, hsa-miR-515-3p, hsa-miR-515-5p, hsa-miR-516a-3p, hsa-miR-516a-5p, hsa-miR-516b, hsa-miR-517*, hsa-miR-517a, hsa-miR-517b, hsa-miR-517c, hsa-miR-518a-3p, hsa-miR-518a-5p, hsa-miR-518b, hsa-miR-518c, hsa-miR-518c*, hsa-miR-518d-3p, hsa-miR-518d-5p, hsa-miR-518c, hsa-miR-518c*, hsa-miR-518f, hsa-miR-518f*, hsa-miR-519a, hsa-miR-519b-3p, hsa-miR-519c-3p, hsa-miR-519d, hsa-miR-519c, hsa-miR-519c*, hsa-miR-520a-3p, hsa-miR-520a-5p, hsa-miR-520b, hsa-miR-520c-3p, hsa-miR-520d-3p, hsa-miR-520d-5p, hsa-miR-520c, hsa-miR-520f, hsa-miR-520g, hsa-miR-520h, hsa-miR-521, hsa-miR-522, hsa-miR-523, hsa-miR-524-3p, hsa-miR-524-5p, hsa-miR-525-3p, hsa-miR-525-5p, hsa-miR-526b, hsa-miR-526b*, hsa-miR-532-3p, hsa-miR-532-5p, hsa-miR-539, hsa-miR-541, hsa-miR-541*, hsa-miR-542-3p, hsa-miR-542-5p, hsa-miR-543, hsa-miR-544, hsa-miR-545, hsa-miR-545*, hsa-miR-548a-3p, hsa-miR-548a-5p, hsa-miR-548b-3p, hsa-miR-548b-5p, hsa-miR-548c-3p, hsa-miR-548c-5p, hsa-miR-548d-3p, hsa-miR-548d-5p, hsa-miR-548c, hsa-miR-548f, hsa-miR-548g, hsa-miR-548h, hsa-miR-548i, hsa-miR-548j, hsa-miR-548k, hsa-miR-548l, hsa-miR-548m, hsa-miR-548n, hsa-miR-5480, hsa-miR-548p, hsa-miR-549, hsa-miR-550, hsa-miR-550*, hsa-miR-551a, hsa-miR-551b, hsa-miR-551b*, hsa-miR-552, hsa-miR-553, hsa-miR-554, hsa-miR-555, hsa-miR-556-3p, hsa-miR-556-5p, hsa-miR-557, hsa-miR-558, hsa-miR-559, hsa-miR-561, hsa-miR-562, hsa-miR-563, hsa-miR-564, hsa-miR-566, hsa-miR-567, hsa-miR-568, hsa-miR-569, hsa-miR-570, hsa-miR-571, hsa-miR-572, hsa-miR-573, hsa-miR-574-3p, hsa-miR-574-5p, hsa-miR-575, hsa-miR-576-3p, hsa-miR-576-5p, hsa-miR-577, hsa-miR-578, hsa-miR-579, hsa-miR-580, hsa-miR-581, hsa-miR-582-3p, hsa-miR-582-5p, hsa-miR-583, hsa-miR-584, hsa-miR-585, hsa-miR-586, hsa-miR-587, hsa-miR-588, hsa-miR-589, hsa-miR-589*, hsa-miR-590-3p, hsa-miR-590-5p, hsa-miR-591, hsa-miR-592, hsa-miR-593, hsa-miR-593*, hsa-miR-595, hsa-miR-596, hsa-miR-597, hsa-miR-598, hsa-miR-599, hsa-miR-600, hsa-miR-601, hsa-miR-602, hsa-miR-603, hsa-miR-604, hsa-miR-605, hsa-miR-606, hsa-miR-607, hsa-miR-608, hsa-miR-609, hsa-miR-610, hsa-miR-611, hsa-miR-612, hsa-miR-613, hsa-miR-614, hsa-miR-615-3p, hsa-miR-615-5p, hsa-miR-616, hsa-miR-616*, hsa-miR-617, hsa-miR-618, hsa-miR-619, hsa-miR-620, hsa-miR-621, hsa-miR-622, hsa-miR-623, hsa-miR-624, hsa-miR-624*, hsa-miR-625, hsa-miR-625*, hsa-miR-626, hsa-miR-627, hsa-miR-628-3p, hsa-miR-628-5p, hsa-miR-629, hsa-miR-629*, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-653, hsa-miR-654-3p, hsa-miR-654-5p, hsa-miR-655, hsa-miR-656, hsa-miR-657, hsa-miR-658, hsa-miR-659, hsa-miR-660, hsa-miR-661, hsa-miR-662, hsa-miR-663, hsa-miR-663b, hsa-miR-664, hsa-miR-664*, hsa-miR-665, hsa-miR-668, hsa-miR-671-3p, hsa-miR-671-5p, hsa-miR-675, hsa-miR-7, hsa-miR-708, hsa-miR-708*, hsa-miR-7-1*, hsa-miR-7-2*, hsa-miR-720, hsa-miR-744, hsa-miR-744*, hsa-miR-758, hsa-miR-760, hsa-miR-765, hsa-miR-766, hsa-miR-767-3p, hsa-miR-767-5p, hsa-miR-768-3p, hsa-miR-768-5p, hsa-miR-769-3p, hsa-miR-769-5p, hsa-miR-770-5p, hsa-miR-802, hsa-miR-873, hsa-miR-874, hsa-miR-875-3p, hsa-miR-875-5p, hsa-miR-876-3p, hsa-miR-876-5p, hsa-miR-877, hsa-miR-877*, hsa-miR-885-3p, hsa-miR-885-5p, hsa-miR-886-3p, hsa-miR-886-5p, hsa-miR-887, hsa-miR-888, hsa-miR-888*, hsa-miR-889, hsa-miR-890, hsa-miR-891a, hsa-miR-891b, hsa-miR-892a, hsa-miR-892b, hsa-miR-9, hsa-miR-9*, hsa-miR-920, hsa-miR-921, hsa-miR-922, hsa-miR-923, hsa-miR-924, hsa-miR-92a, hsa-miR-92a-1*, hsa-miR-92a-2*, hsa-miR-92b, hsa-miR-92b*, hsa-miR-93, hsa-miR-93*, hsa-miR-933, hsa-miR-934, hsa-miR-935, hsa-miR-936, hsa-miR-937, hsa-miR-938, hsa-miR-939, hsa-miR-940, hsa-miR-941, hsa-miR-942, hsa-miR-943, hsa-miR-944, hsa-miR-95, hsa-miR-96, hsa-miR-96*, hsa-miR-98, hsa-miR-99a, hsa-miR-99a*, hsa-miR-99b, and hsa-miR-99b*.

A miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (derepress the polypeptide). In one embodiment, derepression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, an small interfering nucleic acid that is substantially complementary to a miRNA is one that is capable of hybridizing with a miRNA, and blocking the miRNA's activity. In some embodiments, an small interfering nucleic acid that is substantially complementary to a miRNA is an small interfering nucleic acid that is complementary with the miRNA at all but 1, 2, 3, 4, 5, 6, 7, 8, 9, 10, 11, 12, 13, 14, 15, 16, 17, or 18 bases. In some embodiments, an small interfering nucleic acid sequence that is substantially complementary to a miRNA, is an small interfering nucleic acid sequence that is complementary with the miRNA at, at least, one base.

A “miRNA Inhibitor” is an agent that blocks miRNA function, expression and/or processing. For instance, these molecules include but are not limited to microRNA specific antisense, microRNA sponges, tough decoy RNAs (TuD RNAs) and microRNA oligonucleotides (double-stranded, hairpin, short oligonucleotides) that inhibit miRNA interaction with a Drosha complex. MicroRNA inhibitors can be expressed in cells from a transgenes of a nucleic acid, as discussed above. MicroRNA sponges specifically inhibit miRNAs through a complementary heptameric seed sequence (Ebert, M. S. Nature Methods, Epub Aug. 12, 2007;). In some embodiments, an entire family of miRNAs can be silenced using a single sponge sequence. TuD RNAs achieve efficient and long-term-suppression of specific miRNAs in mammalian cells (See, e.g., Takeshi Haraguchi, et al., Nucleic Acids Research, 2009, Vol. 37, No. 6 e43, the contents of which relating to TuD RNAs are incorporated herein by reference). Other methods for silencing miRNA function (derepression of miRNA targets) in cells will be apparent to one of ordinary skill in the art.

In some embodiments, nucleic acids described herein (e.g., a nucleic acid having an ITR comprising any one of SEQ ID NOs: 1-4) may be useful for delivering gene editing molecules (e.g., nucleases) to a subject. In some embodiments a nucleic acid described by the disclosure comprises a heterologous nucleic acid insert encodes a nuclease. As used herein, the terms “endonuclease” and “nuclease” refer to an enzyme that cleaves a phosphodiester bond or bonds within a polynucleotide chain. Nucleases may be naturally occurring or genetically engineered. Genetically engineered nucleases are particularly useful for genome editing and are generally classified into four families: zinc finger nucleases (ZFNs), transcription activator-like effector nucleases (TALENs), engineered meganucleases and CRISPR-associated proteins (Cas nucleases). In some embodiments, the nuclease is a ZFN. In some embodiments, the ZFN comprises a FokI cleavage domain. In some embodiments, the ZFN comprises Cys2His2 fold group. In some embodiments, the nuclease is a TALEN. In some embodiments, the TALEN comprises a FokI cleavage domain. In some embodiments, the nuclease is an engineered meganuclease.

The term “CRISPR” refers to “clustered regularly interspaced short palindromic repeats”, which are DNA loci containing short repetitions of base sequences. CRISPR loci form a portion of a prokaryotic adaptive immune system that confers resistance to foreign genetic material. Each CRISPR loci is flanked by short segments of “spacer DNA”, which are derived from viral genomic material. In the Type II CRISPR system, spacer DNA hybridizes to transactivating RNA (tracrRNA) and is processed into CRISPR-RNA (crRNA) and subsequently associates with CRISPR-associated nucleases (Cas nucleases) to form complexes that recognize and degrade foreign DNA. In certain embodiments, the nuclease is a CRISPR-associated nuclease (Cas nuclease). Examples of CRISPR nucleases include, but are not limited to Cas9, Cas6 and dCas9. dCas9 is an engineered Cas protein that binds to a target locus but does not cleave said locus. In some embodiments, the nuclease is Cas9. In some embodiments, the Cas9 is derived from the bacteria S. pyogenes (SpCas9).

For the purpose of genome editing, the CRISPR system can be modified to combine the tracrRNA and crRNA in to a single guide RNA (sgRNA) or just (gRNA). As used herein, the term “guide RNA” or “gRNA” refers to a polynucleotide sequence that is complementary to a target sequence in a cell and associates with a Cas nuclease, thereby directing the Cas nuclease to the target sequence. In some embodiments, a nucleic acid described by the disclosure comprises a heterologous nucleic acid insert encoding a guide RNA (gRNA). In some embodiments, a gRNA ranges between 1 and 30 nucleotides in length. In some embodiments, a gRNA ranges between 5 and 25 nucleotides in length. In some embodiments, a gRNA ranges between 10 and 20 nucleotides in length. In some embodiments, a gRNA ranges between 14 and 18 nucleotides in length. In some embodiments, a nucleic acid described by the disclosure comprises a heterologous nucleic acid insert encoding a gRNA and a CRISPR nuclease.

In some aspects, the disclosure relates to a nucleic acid encoding a heterologous nucleic acid insert that does not encode a functional protein. For example, in the context of gene therapy, transgene promoter integration may cause oncogene activation. Accordingly, in some embodiments, the disclosure relates to a heterologous nucleic acid insert encoding a promoterless construct. Without wishing to be bound by any particular theory, a promoterless expression construct is useful, in some embodiments, as a substrate for gene editing.

As used herein, “genome editing” refers to adding, disrupting or changing genomic sequences (e.g., a gene sequence). In some embodiments, genome editing is performed using engineered proteins and related molecules. In some aspects, genome editing comprises the use of engineered nucleases to cleave a target genomic locus. In some embodiments, genome editing further comprises inserting, deleting, mutating or substituting nucleic acid residues at a cleaved locus. In some embodiments, inserting, deleting, mutating or substituting nucleic acid residues at a cleaved locus is accomplished through endogenous cellular mechanisms such as homologous recombination (HR) and non-homologous end joining (NHEJ). Exemplary genome editing technologies include, but are not limited to Transcription Activator-like Effector Nucleases (TALENs), Zinc Finger Nucleases (ZFNs), engineered meganuclease re-engineered homing endonucleases, the CRISPR/Cas system. In some embodiments, the gene editing technologies are proteins or molecules related to TALENs, including but not limited to transcription activator-like effectors (TALEs) and restriction endonucleases (e.g., FokI). In some embodiments, the gene editing technologies are proteins or molecules related to ZFNs, including but not limited to proteins comprising the Cys2His2 fold group (for example Zif268 (EGR1)), and restriction endonucleases (e.g., FokI). In some embodiments, the gene editing technologies are proteins or molecules related to the CRISPR/Cas system, including but not limited to Cas9, Cas6, dCas9, CRISPR RNA (crRNA) and trans-activating crRNA (tracrRNA). In some embodiments, the promoterless construct provides a substrate for TALENS, zinc finger nucleases (ZFNs), meganucleases, Cas9, and other gene editing proteins.

In some aspects, the disclosure relates to a nucleic acid encoding a heterologous nucleic acid insert that encodes a DNA or mRNA vaccine. As used herein, “DNA vaccine” or “mRNA vaccine” refers to a nucleic acid encoding an antigen that stimulates an immune response (e.g., a cellular immune response or a humoral immune response) against that antigen in a host. In some embodiments, the immune response is a protective response that protects against a future infection or condition. However, in some embodiments, the immune response treats (e.g., eradicates or attenuates) an existing infection or condition. Examples of DNA vaccines include HL chain Ig, scFv, single-domain Ig derived from Camelidae (VhH) or cartilaginous fish (Vnar), nanobody, and other paratope recognitions peptides and fusion peptides collectively referred to as Ig (or Ig-like) molecules.

In some embodiments, a heterologous nucleic acid insert encodes an Ig or Ig-like molecule. In some embodiments, the Ig (or Ig-like) molecules are unmodified protein sequences derived from monoclonal antibody sequences. In some embodiments, the Ig (or Ig-like) molecules are unmodified protein sequences derived from murine or other mammalian monoclonal antibody sequences.

In some embodiments, the Ig (or Ig-like) molecules are unmodified protein sequences derived from synthetic randomly generated peptide libraries. In some embodiments, the libraries were derived from complimentary DNA obtained from naïve vertebrate species. The species include, but are not limited to mammals, such as primates (e.g., humans and non-human primates), rodents (e.g., mouse, rats), ungulates, camelids, equines, canines, felines, marsupials, and animals of agricultural interest; Avian species, including chickens, ducks, and geese; piscine species including cartilaginous fish, lamprey eels, and jawed fish species.

In some embodiments, the heterologous nucleic acid encodes the heavy and light chains for an immunoglobulin (Ig), such that when administered to a permissive cell, an assembled Ig is secreted into the circulatory system. In some embodiments, the Ig molecule is not secreted and acts internally as a so-called “intra-body”. In some embodiments, a heterologous nucleic acid insert encodes an Ig molecule that is an engineered single-chain antibody consisting of the heavy and light chain variable regions in one polypeptide (scFv). The scFv retains avidity and specificity for the target antigen.

In some embodiments, a heterologous nucleic acid insert encodes an Ig molecule that binds to microbial agents and affects the infectivity of the microbe. In some embodiments, the microbial agents are prokaryotic organisms. In some embodiments, the microbial agents are rickettsia, mycoplasma, or other intracellular life forms.

In some embodiments, a heterologous nucleic acid insert encodes an Ig molecule that binds to viral structural protein(s) of human pathogenic viruses, including but not limited an Ebola virus viral protein, a human immune deficiency viral protein, a papilloma viral protein, a herpes simplex 1 viral protein, a herpes simplex 2 viral protein, a HCV A viral protein, a HCV B viral protein, a HCV C viral protein, a HCV non-A viral protein, a HCV non-B viral protein, or a dengue hemorrhagic fever viral protein. In some embodiments, a heterologous nucleic acid insert encodes an Ig molecule that binds to viral structural protein(s) of a zoonotic pathogen, including, but not limited to foot and mouth disease virus and rabies virus.

In some aspects, the disclosure relates to isolated nucleic acids comprising a transgene encoding one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in a 3′ untranslated region (3′ UTR) of a transgene, for example between the last codon of a nucleic acid sequence encoding a therapeutic protein or variant thereof, and a poly A sequence.

In some embodiments, a transgene comprises one or more (e.g., 1, 2, 3, 4, 5, or more) miRNA binding sites that de-target expression of the therapeutic protein from liver cells. For example, in some embodiments, a transgene comprises one or more miR-122 binding sites. In some embodiments, a transgene comprises one or more miR-142 binding sites.

In some embodiments, a transgene comprises one or more (e.g., 1, 2, 3, 4, 5, or more) miRNA binding sites that de-target expression of therapeutic proteins from immune cells (e.g., antigen presenting cells (APCs), such as macrophages, dendrites, etc.). Incorporation of miRNA binding sites for immune-associated miRNAs may de-target transgene (e.g., one or more therapeutic proteins) expression from antigen presenting cells and thus reduce or eliminate immune responses (cellular and/or humoral) produced in the subject against products of the transgene, for example as described in US 2018/0066279, the entire contents of which are incorporated herein by reference.

As used herein an “immune-associated miRNA” is a miRNA preferentially expressed in a cell of the immune system, such as an antigen presenting cell (APC). In some embodiments, an immune-associated miRNA is an miRNA expressed in immune cells that exhibits at least a 2-fold, 3-fold, 4-fold, 5-fold, 6-fold, 7-fold, 8-fold, 9-fold, 10-fold higher level of expression in an immune cell compared with a non-immune cell (e.g., a control cell, such as a HeLa cell, HEK293 cell, mesenchymal cell, etc.). In some embodiments, the cell of the immune system (immune cell) in which the immune-associated miRNA is expressed is a B cell, T cell, Killer T cell, Helper T cell, γδT cell, dendritic cell, macrophage, monocyte, vascular endothelial cell or other immune cell. In some embodiments, the cell of the immune system is a B cell expressing one or more of the following markers: B220, BLAST-2 (EBVCS), Bu-1, CD19, CD20 (L26), CD22, CD24, CD27, CD57, CD72, CD79a, CD79b, CD86, chB6, D8/17, FMC7, L26, M17, MUM-1, Pax-5 (BSAP), and PC47H. In some embodiments, the cell of the immune system is a T cell expressing one or more of the following markers: ART2, CD1a, CD1d, CD11b (Mac-1), CD134 (OX40), CD150, CD2, CD25 (interleukin 2 receptor alpha), CD3, CD38, CD4, CD45RO, CD5, CD7, CD72, CD8, CRTAM, FOXP3, FT2, GPCA, HLA-DR, HML-1, HT23A, Leu-22, Ly-2, Ly-m22, MICG, MRC OX 8, MRC OX-22, OX40, PD-1 (Programmed death-1), RT6, TCR (T cell receptor), Thy-1 (CD90), and TSA-2 (Thymic shared Ag-2). In some embodiments, the immune-associated miRNA is selected from: miR-15a, miR-16-1, miR-17, miR-18a, miR-19a, miR-19b-1, miR-20a, miR-21, miR-29a/b/c, miR-30b, miR-31, miR-34a, miR-92a-1, miR-106a, miR-125a/b, miR-142-3p, miR-146a, miR-150, miR-155, miR-181a, miR-223 and miR-424, miR-221, miR-222, let-7i, miR-148, and miR-152. In some embodiments, a transgene described herein comprises one or more binding sites for miR-142

In some aspects, nucleic acids described by the disclosure are useful for the production of modified cells, such as ex vivo modified cells. As used herein, “ex vivo modified cell” refers to a cell (e.g., a mammalian cell) that is removed from a subject, genetically modified (e.g., transfected or transduced with exogenous nucleic acids, or genetically reprogrammed), cultured or expanded, and optionally, returned to a subject (e.g., either the same subject, or a different subject). Generally, ex vivo modified cells are useful for autologous cell therapy, or allogeneic cell therapy. For example, cells may be removed from a subject having a disease associated with a particular genetic defect (e.g., overexpression of a particular protein), transfected with a nucleic acid that corrects the genetic defect (e.g. reduces expression of the protein), and reintroduced into the subject. In another non-limiting example, cells are removed from a subject, genetically reprogrammed (e.g., dedifferentiated or transdifferentiated into stem cells), expanded, and reintroduced into the subject. In some embodiments, ex vivo modified cells produced by transfection with a nucleic acid as described by the disclosure have an improved safety profile compared to ex vivo cells produced by currently available gene therapy vectors.

In some aspects, nucleic acids described by the disclosure are useful for the production of chimeric antigen T-cells (CARTs). Chimeric Antigen Receptors (CARs) are engineered T cell receptors displaying specificity against target antigens based on a single chain FV (scFv) antibody moiety. Generally, CARTs are produced by transduction of T-cells with lentiviral vectors comprising DNA encoding CARs. Lentiviral transduction, in some embodiments, raises the risk of insertional mutagenesis leading to cancer. As described by the disclosure, nucleic acids having asymmetric interrupted self-complementary sequences exhibit reduced likelihood of insertional mutagenesis compared to other gene therapy modalities. Accordingly, in some embodiments a nucleic acid described by the disclosure comprises a heterologous nucleic acid insert (e.g., a transgene) encoding a CAR. In some embodiments, a CART produced by transduction with a nucleic acid described by the disclosure exhibits an improved safety profile compared to a CART produced by lentiviral transduction.

The nucleic acid, preferably suspended in a physiologically compatible carrier (i.e., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the nucleic acids (e.g., a nucleic acid having an ITR comprising any one of SEQ ID NOs: 1-4) to a mammalian subject may be by, for example, intramuscular injection or by administration into the bloodstream of the mammalian subject. Administration into the bloodstream may be by injection into a vein, an artery, or any other vascular conduit. In some embodiments, the nucleic acids are administered into the bloodstream by way of isolated limb perfusion, a technique well known in the surgical arts, the method essentially enabling the artisan to isolate a limb from the systemic circulation prior to administration of the nucleic acid. A variant of the isolated limb perfusion technique, described in U.S. Pat. No. 6,177,403, can also be employed by the skilled artisan to administer the nucleic acid(s) into the vasculature of an isolated limb to potentially enhance transfection of muscle cells or tissue. Moreover, in certain instances, it may be desirable to deliver the nucleic acid(s) to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000).

Suitable carriers may be readily selected by one of skill in the art in view of the indication for which the nucleic acid(s) is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the nucleic acid(s) and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The nucleic acid(s) are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

The dose of nucleic acid(s) required to achieve a particular “therapeutic effect,” will vary based on several factors including, but not limited to: the route of nucleic acid administration, the level of gene or RNA expression required to achieve a therapeutic effect, the specific disease or disorder being treated, and the stability of the gene or RNA product. One of skill in the art can readily determine a nucleic acid dose range to treat a patient having a particular disease or disorder based on the aforementioned factors, as well as other factors that are well known in the art.

Dosage regime may be adjusted to provide the optimum therapeutic response. For example, the oligonucleotide may be repeatedly administered, e.g., several doses may be administered daily or the dose may be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the nucleic acid-based therapeutic constructs in suitably formulated pharmaceutical compositions disclosed herein either subcutaneously, intraopancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver nucleic acids. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the nucleic acid in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The nucleic acid compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the nucleic acids may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 ANG., containing an aqueous solution in the core.

In some embodiments, a liposome comprises cationic lipids. The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In some embodiments, cationic lipids include saturated and unsaturated alkyl and alicyclic ethers and esters of amines, amides, or derivatives thereof. In some embodiments, cationic lipids comprise straight-chain, branched alkyl, alkenyl groups, or any combination of the foregoing. In some embodiments, cationic lipids contain from 1 to about 25 carbon atoms (e.g., 1, 2, 3, 4, 5, 6, 7, 8, 9, 10, 11, 12, 13, 14, 15, 16, 17, 18, 19, 20, 21, 22, 23, 24, or 25 carbon atoms. In some embodiments, cationic lipids contain more than 25 carbon atoms. In some embodiments, straight chain or branched alkyl or alkene groups have six or more carbon atoms. A cationic lipid may also comprise, in some embodiments, one or more alicyclic groups. Non-limiting examples of alicyclic groups include cholesterol and other steroid groups. In some embodiments, cationic lipids are prepared with a one or more counterions. Examples of counterions (anions) include but are not limited to Cl—, Br—, I—, F—, acetate, trifluoroacetate, sulfate, nitrite, and nitrate.

Non-limiting examples of cationic lipids include polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N-[1-(2,3-dioleoyloxy)-propyl]-N,N,N-trimethylammonium chloride (DOTMA), N-[1-(2,3-dioleoyloxy)-propyl]-N,N,N-trimethylammonium methylsulfate (DOTAP), 3β-[N-(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). Nucleic acids (e.g., a nucleic acid having an ITR comprising any one of SEQ ID NOs: 1-4) can also be complexed with, e.g., poly (L-lysine) or avidin and lipids may, or may not, be included in this mixture, e.g., steryl-poly (L-lysine).

In some embodiments, a nucleic acid described by the disclosure is delivered using a cationic lipid described in U.S. Pat. No. 8,158,601, or a polyamine compound or lipid as described in U.S. Pat. No. 8,034,376, the contents of each of which are incorporated herein by reference.

In some embodiments, a nucleic acid described by the disclosure is conjugated (e.g., covalently bound to an agent that increases cellular uptake. An “agent that increases cellular uptake” is a molecule that facilitates transport of a nucleic acid across a lipid membrane. For example, a nucleic acid may be conjugated to a lipophilic compound (e.g., cholesterol, tocopherol, etc.), a cell penetrating peptide (CPP) (e.g., penetratin, TAT, Syn1B, etc.), and polyamines (e.g., spermine). Further examples of agents that increase cellular uptake are disclosed, for example, in Winkler (2013). Oligonucleotide conjugates for therapeutic applications. Ther. Deliv. 4(7); 791-809, the contents of which are incorporated herein by reference.

In some embodiments, a nucleic acid described by the disclosure is conjugated to a polymer (e.g., a polymeric molecule) or a folate molecule (e.g., folic acid molecule). Generally, delivery of nucleic acids conjugated to polymers is known in the art, for example as described in WO2000/34343 and WO2008/022309, the contents of which are incorporated herein by reference. In some embodiments, a nucleic acid described by the disclosure is conjugated to a poly(amide) polymer, for example as described by U.S. Pat. No. 8,987,377. In some embodiments, a nucleic acid described by the disclosure is conjugated to a folic acid molecule as described in U.S. Pat. No. 8,507,455, the contents of which are incorporated herein by reference.

In some embodiments, a nucleic acid described by the disclosure is conjugated to a carbohydrate, for example as described in U.S. Pat. No. 8,450,467, the contents of which are incorporated herein by reference.

Alternatively, nanocapsule formulations of the nucleic acid may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In some embodiments, a nucleic acid described by the disclosure is delivered by a lipid nanoparticle. Generally, lipid nanoparticles comprise an ionizable amino lipid (e.g., heptatriaconta-6,9,28,31-tetraen-19-yl 4-(dimethylamino)butanoate, DLin-MC3-DMA, a phosphatidylcholine (1,2-distearoyl-sn-glycero-3-phosphocholine, DSPC), cholesterol and a coat lipid (polyethylene glycol-dimyristolglycerol, PEG-DMG), for example as disclosed by Tam et al. (2013). Advances in Lipid Nanoparticles for siRNA delivery. Pharmaceuticals 5(3): 498-507. In some embodiments, a lipid nanoparticle has a mean diameter between about 10 and about 1000 nm. In some embodiments, a lipid nanoparticle has a diameter that is less than 300 nm. In some embodiments, a lipid nanoparticle has a diameter between about 10 and about 300 nm. In some embodiments, a lipid nanoparticle has a diameter that is less than 200 nm. In some embodiments, a lipid nanoparticle has a diameter between about 25 and about 200 nm. In some embodiments, a lipid nanoparticle preparation (e.g., composition comprising a plurality of lipid nanoparticles) has a size distribution in which the mean size (e.g., diameter) is about 70 nm to about 200 nm, and more typically the mean size is about 100 nm or less.

In some embodiments, a nucleic acid described by the disclosure is delivered by a gold nanoparticle. Generally, a nucleic acid can be covalently bound to a gold nanoparticle or non-covalently bound to a gold nanoparticle (e.g., bound by a charge-charge interaction), for example as described by Ding et al. (2014). Gold Nanoparticles for Nucleic Acid Delivery. Mol. Ther. 22(6); 1075-1083. In some embodiments, gold nanoparticle-nucleic acid conjugates are produced using methods described, for example, in U.S. Pat. No. 6,812,334, the contents of which are incorporated herein by reference.

In addition to the methods of delivery described above, the following techniques are also contemplated as alternative methods of delivering the nucleic acid compositions to a host. Sonophoresis (e.g., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some embodiments, the disclosure provides a method of delivering a heterologous nucleic acid to a cell (e.g., a host cell), the method comprising delivering to the cell a nucleic acid as described by the disclosure.

In some aspects, the disclosure provides transfected host cells. A “host cell” refers to any cell that harbors, or is capable of harboring, a substance of interest. A host cell may be used as a recipient of a nucleic acid as described by the disclosure. The term includes the progeny of the original cell which has been transfected. Thus, a “host cell” as used herein may refer to a cell which has been transfected with an exogenous DNA sequence (e.g., a nucleic acid as described by the disclosure). It is understood that the progeny of a single parental cell may not necessarily be completely identical in morphology or in genomic or total DNA complement as the original parent, due to natural, accidental, or deliberate mutation.

In some embodiments, a host cell is a bacterial cell. In some embodiments, a host cell is a HEK293 cell or is derived from a HEK293 cell. In some embodiments, a host cell is a permissive cell. In some embodiments, a host cell is not a permissive cell. Often a host cell is a mammalian cell. In some aspects, the disclosure provides a method of delivering a heterologous nucleic acid to a subject comprising administering a host cell having a nucleic acid as described by the disclosure to the subject. For example, in some embodiments, a host cell is a blood cell, such as a human blood cell, comprising a nucleic acid as described by the disclosure (e.g., a nucleic acid having a heterologous nucleic acid insert encoding a blood disease-associated transgene). Without wishing to be bound by any particular theory, delivery of such a host cell is useful, in some embodiments, for treatment of a disease or disorder.

Aspects of the disclosure relate to the discovery that nucleic acids as described herein elicit a reduced immune response (e.g., do not elicit an immune response) in a host relative to currently used viral and bacterially-derived gene therapy vectors. In some aspects, the disclosure provides a method of delivering a heterologous nucleic acid to a subject, the method comprising delivering to the subject a nucleic acid as described by the disclosure, wherein the delivery of the nucleic acid does not result in an immune response against the nucleic acid in the subject. In some embodiments, the immune response is a humoral response. Humoral immune response refers to production of antigen-specific antibodies by B lymphocytes. In some embodiments, the immune response is a cellular response. A cellular immune response refers to an immune response that does not involve antibodies but rather activation of immune cells (e.g., phagocytes, antigen-specific T-cells, macrophages, natural killer cells, etc.) by an antigen (e.g., an exogenous nucleic acid).

Without wishing to be bound by any particular theory, the lack of immune response elicited by administration of nucleic acids as described by the disclosure allows the nucleic acids to be administered to a host on multiple occasions. In some embodiments, the number of occasions in which a heterologous nucleic acid is delivered to a subject is in a range of 2 to 10 times (e.g., 2, 3, 4, 5, 6, 7, 8, 9, or 10 times). In some embodiments, a heterologous nucleic acid is delivered to a subject more than 10 times.

In some embodiments, a dose of nucleic acid (e.g., a nucleic acid having an ITR comprising any one of SEQ ID NOs: 1-4) is administered to a subject no more than once per calendar day (e.g., a 24-hour period). In some embodiments, a dose of nucleic acid is administered to a subject no more than once per 2, 3, 4, 5, 6, or 7 calendar days. In some embodiments, a dose of nucleic acid is administered to a subject no more than once per calendar week (e.g., 7 calendar days). In some embodiments, a dose of nucleic acid is administered to a subject no more than bi-weekly (e.g., once in a two calendar week period). In some embodiments, a dose of nucleic acid is administered to a subject no more than once per calendar month (e.g., once in 30 calendar days). In some embodiments, a dose of nucleic acid is administered to a subject no more than once per six calendar months. In some embodiments, a dose of nucleic acid is administered to a subject no more than once per calendar year (e.g., 365 days or 366 days in a leap year).

As disclosed herein nucleic acids (including DNA expression constructs that may be used to express them) may be administered by any suitable route. For use in therapy, an effective amount of the nucleic acid (e.g., oligonucleotide) and/or other therapeutic agent can be administered to a subject by any mode that delivers the agent to the desired tissue, e.g., muscle tissue. In some embodiments, agents (e.g., nucleic acids) are administered intramuscularly. Other suitable routes of administration include but are not limited to oral, parenteral, intravenous, intraperitoneal, intranasal, sublingual, intratracheal, inhalation, subcutaneous, ocular, vaginal, and rectal. Systemic routes include oral and parenteral. Several types of devices are regularly used for administration by inhalation. These types of devices include metered dose inhalers (MDI), breath-actuated MDI, dry powder inhaler (DPI), spacer/holding chambers in combination with MDI, and nebulizers.

For oral administration, the agents can be formulated readily by combining the active compound(s) with pharmaceutically acceptable carriers well known in the art. Such carriers enable the agents of the disclosure to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally oral formulations may also be formulated in saline or buffers for neutralizing internal acid conditions or may be administered without any carriers.

Pharmaceutical preparations that can be used orally include push fit capsules made of gelatin, as well as soft, scaled capsules made of gelatin and a plasticizer, such as glycerol or sorbitol. The push fit capsules can contain the active ingredients in admixture with filler such as lactose, binders such as starches, and/or lubricants such as talc or magnesium stearate and, optionally, stabilizers. In soft capsules, the active agents may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. Formulations for oral administration are typically in dosages suitable for such administration.

For buccal administration, the compositions may take the form of tablets or lozenges formulated in conventional manner.

For administration by inhalation, agents (e.g., nucleic acids) for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The agents (e.g., nucleic acids), when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of agents (e.g., antisense nucleic acids) in water-soluble form. Additionally, suspensions of agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the agents to allow for the preparation of highly concentrated solutions. Alternatively, agents (e.g., nucleic acids) may be in powder form for constitution with a suitable vehicle, e.g., sterile pyrogen-free water, before use. Agents (e.g., nucleic acids) may also be formulated in rectal or vaginal compositions such as suppositories or retention enemas, e.g., containing conventional suppository bases such as cocoa butter or other glycerides.

Other delivery systems can include time-release, delayed release or sustained release delivery systems. Such systems can avoid repeated administrations of the agents (e.g., antisense nucleic acids), increasing convenience to the subject and the physician. Many types of release delivery systems are available. They include polymer base systems such as poly(lactide glycolide), copolyoxalates, polycaprolactones, polyesteramides, polyorthoesters, polyhydroxybutyric acid, and polyanhydrides. Delivery systems also include non-polymer systems that are: lipids including sterols such as cholesterol, cholesterol esters and fatty acids or neutral fats such as mono, di, and tri glycerides; hydrogel release systems; silastic systems; peptide-based systems; wax coatings; compressed tablets using conventional binders and excipients; partially fused implants; and others disclosed herein.

### EXAMPLES

### Example 1

This example describes newly discovered ITR8 and ITRrh.39 sequences that are structurally and functionally distinct from previously described ITRs. In some embodiments, novel ITRs disclosed herein (or rAAVs comprising novel ITRs or combinations of novel ITRs and previously described ITRs, such as AAV2 ITR) can be used to develop new “hybrid” ITR vector constructs to improve vector production, manufacturing, and in vivo performance. Additionally, these new ITR sequences can be used in combination with different AAV rep and cap genes to develop the best combinations for efficient rAAV vector production.

AAV ITRs interact with a number of host proteins and can trigger anti-viral and DNA damage response pathways. AAV vectors based on new hybrid ITR constructs have the potential to dampen host anti-viral and DNA damage-responses.

Without wishing to be bound by any particular theory, rAAVs using hybrid/asymmetric ITR designs may have reduced ability to form intramolecular circular intermediates, with increased efficiency for directional tail-to-head intermolecular recombination of linear genomes capable of expressing dual-vector-encoded trans-spliced gene products, enhancing dual-vector gene delivery, and solving the 4.7-kb packaging limitation inherent to AAV vectors. In some embodiments, the ITRs may generate rAAV genomes with different in vivo episomal stabilities, integration profiles, and alternative genetic fates for different applications. The ITRs may also harbor promoter-like function with cell type-specific activities, and may confer unique/improved transgene expression profiles.

### Example 2

Nucleic acid constructs comprising unique hybrid ITR sequences (combinations of 5′ and 3′ ITR sequences) were designed and tested for transgene expression. The transgene was either enhanced green fluorescent protein (eGFP) or human Alpha-1-antitrypsin (hA1AT). All designed constructs comprised one or more of, in 5′-to-3′ direction, a variable 5′ ITR sequence, a CMV immediate early enhancer, a chicken beta actin (CBA) promoter, a transgene, a rabbit globin poly A sequence, and a variable 3′ ITR sequence.

TABLE 1
Nucleic acid constructs of Example 2 for expression of eGFP

| Description | 5′ ITR sequence | 3′ ITR sequence |
|---|---|---|
| pAAV-2/2-(eGFP) | AAV2 5′ ITR | AAV2 3′ ITR |
| pAAV-2/8-(eGFP) | AAV2 5′ ITR | AAV8 3′ ITR |
| pAAV-2/rh.39-(eGFP) | AAV2 5′ ITR | AAVrh.39 3′ ITR |
| pAAV-8/2-(eGFP) | AAV8 5′ ITR | AAV2 3′ ITR |
| pAAV-8/8-(eGFP) | AAV8 5′ ITR | AAV8 3′ ITR |
| pAAV-8/rh.39-(eGFP) | AAV8 5′ ITR | AAVrh.39 3′ ITR |
| pAAV-rh.39/2-(eGFP) | AAVrh.39 5′ ITR | AAV2 3′ ITR |
| pAAV- rh.39/8-(eGFP) | AAVrh.39 5′ ITR | AAV8 3′ ITR |
| pAAV- rh.39/rh.39-(eGFP) | AAVrh.39 5′ ITR | AAVrh.39 3′ ITR |

Recombinant AAV (rAAV) particles (at a total concentration of 1×10¹¹ vector copies) comprising a nucleic acid construct of Table 1 and an AAV8 capsid protein (e.g., a capsid protein known to target liver, heart and muscle tissues) were systematically injected (via tail vein injection) into 8-week-old mice. The mice were euthanized four weeks after treatment with the rAAV particles and the liver, heart and muscle tissues were removed for testing.

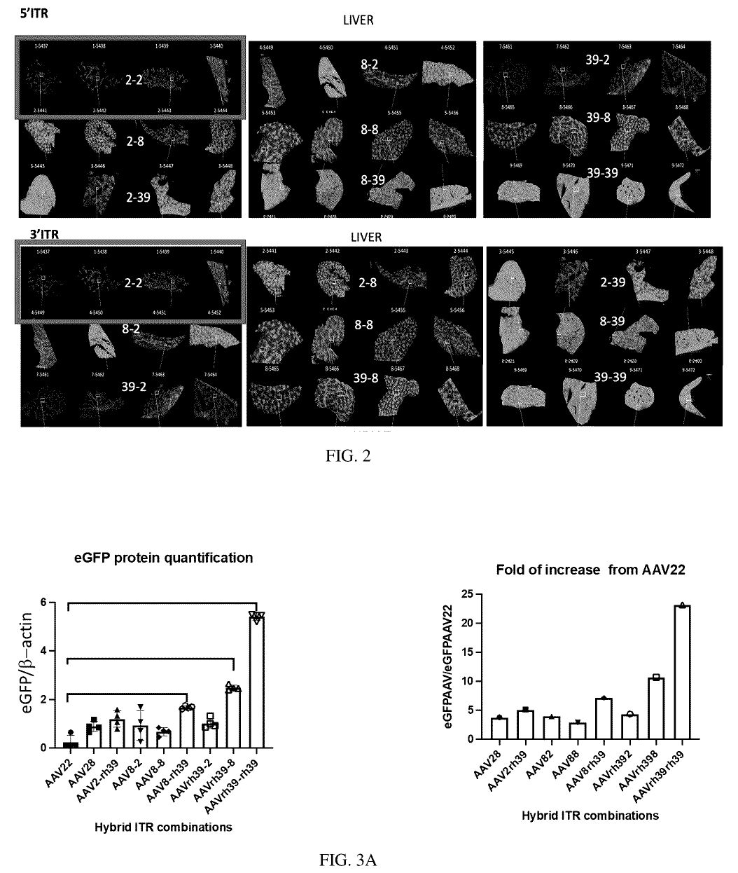

The expression of eGFP in the liver, heart and muscle tissues of mice that were treated with rAAVs comprising the eGFP transgene were qualitatively assessed by fluorescence. As shown in FIG. 2, the hybrid AAV ITR constructs provided increased eGFP reporter expression based on fluorescence compared with the conventional AAV2-2 ITR combination. Notably, the hybrid AAV ITR constructs comprising an AAVrh.39 3′ ITR provided significantly increased expression of eGFP. Similar fluorescence profiles were determined in the heart and muscle tissues of mice that were treated with rAAVs comprising the eGFP transgene.

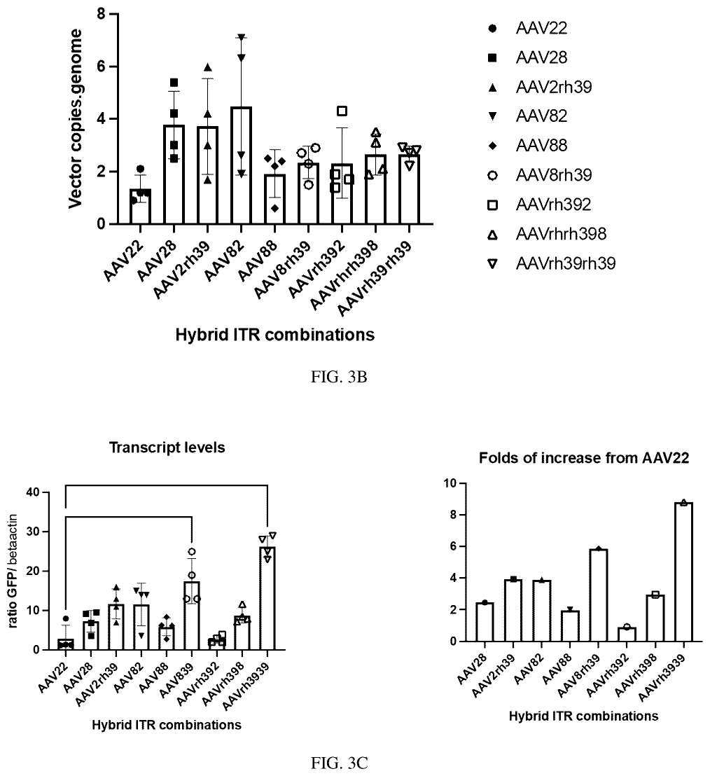

The concentration of eGFP protein expression in the liver was then determined by western blot analysis and normalized to a control protein (β-actin) (FIG. 3A). Mice treated with an rAAV comprising pAAV-2/2-(eGFP) had a very low concentration of eGFP relative to β-actin in their liver tissues. Mice treated with an rAAV comprising a hybrid ITR construct, particularly a hybrid construct comprising an rh.39 ITR (e.g., pAAV-rh.39/8-(eGFP)) had elevated concentrations of eGFP relative to β-actin in their liver tissues compared to mice treated with the AAV2 ITR construct. Interestingly, none of the treated mice had elevated levels of vector copies of the rAAV particles relative to any other mice (FIG. 3B).

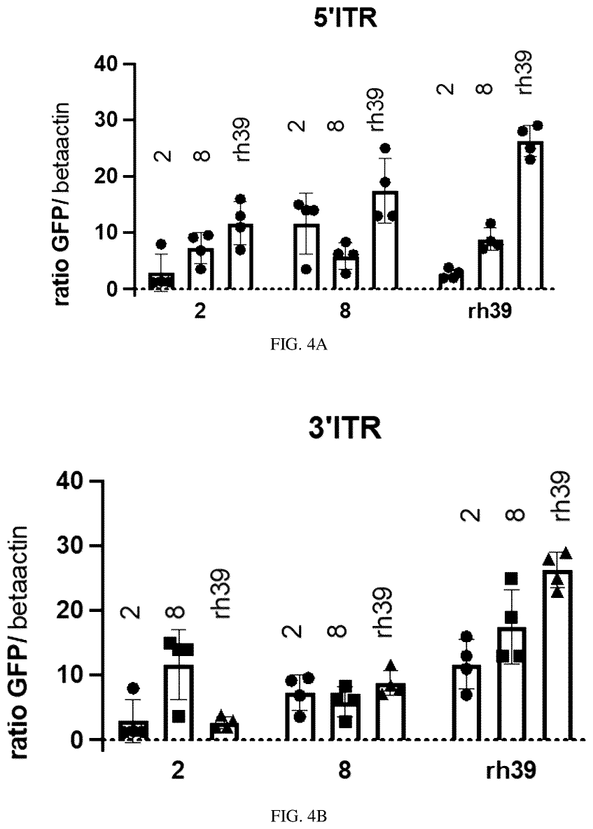

The concentration of eGFP transcript expression in the liver was then determined by ddPCR analysis and normalized to a control protein (β-actin) (FIG. 3C). The concentration of eGFP transcripts shared a similar profile with the observed eGFP protein levels. An analysis of the impact of varying 5′ and 3′ ITR sequences on the transgene expression was also determined using ddPCR data (FIGS. 4A-4B). It was found that inclusion of an AAVrh.39 3′ ITR sequence had a more significant impact on transgene expression (see, FIG. 4B) compared to inclusion of an AAVrh.39 5′ ITR sequence in a hybrid ITR construct.

TABLE 2
Nucleic acid constructs of Example 2 for expression of hA1AT

| Description | 5′ ITR sequence | 3′ ITR sequence |
|---|---|---|
| pAAV-2/2-(hA1AT) | AAV2 5′ ITR | AAV2 3′ ITR |
| pAAV-2/8-(hA1AT) | AAV2 5′ ITR | AAV8 3′ ITR |
| pAAV-2/rh.39-(hA1AT) | AAV2 5′ ITR | AAVrh.39 3′ ITR |
| pAAV-8/2-(hA1AT) | AAV8 5′ ITR | AAV2 3′ ITR |
| pAAV-8/8-(hA1AT) | AAV8 5′ ITR | AAV8 3′ ITR |
| pAAV-8/rh.39-(hA1AT) | AAV8 5′ ITR | AAVrh.39 3′ ITR |
| pAAV-rh.39/2-(hA1AT) | AAVrh.39 5′ ITR | AAV2 3′ ITR |
| pAAV- rh.39/8-(hA1AT) | AAVrh.39 5′ ITR | AAV8 3′ ITR |
| pAAV- rh.39/rh.39-(hA1AT) | AAVrh.39 5′ ITR | AAVrh.39 3′ ITR |

Recombinant AAV (rAAV) particles (at a total concentration of 3×10¹⁰ vector copies) comprising a nucleic acid construct of Table 2 and an AAV8 capsid protein (e.g., a capsid protein known to target liver, heart and muscle tissues) were systematically injected (via tail vein injection) into 8-week-old mice. Blood was drawn from the mice at weeks 1 and 3 following injection for analysis. The mice were euthanized four weeks after treatment with the rAAV particles and the liver, heart and muscle tissues were removed for testing.

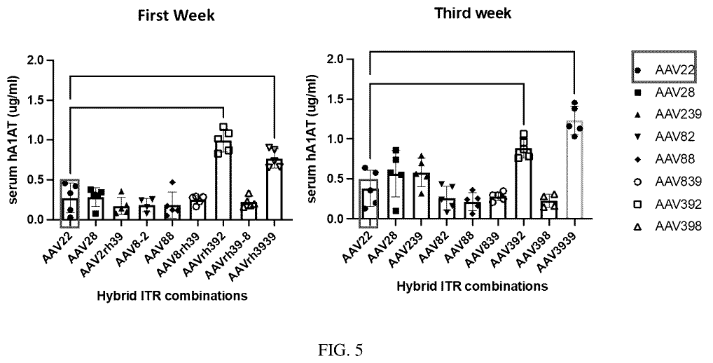

The expression of hA1AT protein in the blood of mice that were treated with rAAVs comprising the hA1AT transgene were assessed by western blot analysis. As shown in FIG. 5, the hybrid AAV ITR constructs of the Example provided stable expression of hA1AT protein for up to three weeks after injection of the AAV particles. In particular, it was found that delivery of nucleic acid constructs that utilized rh.39 ITR sequences such as pAAV-rh.39/2-(hA1AT) (comprising an AAVrh.39 5′ ITR) provided levels of serum hA1AT protein of up to 1.0 μg/mL.

Collectively, these data demonstrate that nucleic acid constructs comprising hybrid ITR sequences (such as those nucleic acid constructs comprising an AAV8 ITR and/or an AAVrh.39 ITR) are capable of providing stable in vivo transgene expression. Furthermore, these nucleic acid constructs are capable of providing stable and increased transgene expression, relative to nucleic acid constructs using conventional ITRs, without a significant increase in vector copy numbers. The nucleic acid constructs comprising hybrid ITR sequences of the disclosure provide novel and effective approaches for transgene delivery.

SEQUENCES

Novel AAV8 3′ ITR (SEQ ID NO: 1)
ggttacccctagtgatggagttggccactccctctatgcgcgctcgctcgctcggtggggccggcagagcagagctctgccgtctgcgga
cctttggtccgcaggccccaccgagcgagcgagcgcgcatagagggagtggccaa
Novel AAV8 5′ ITR (SEQ ID NO: 2)
ttggccactccctctatgcgcgctcgctcgctcggtggggcctgcggaccaaaggtccgcagacggcagagctctgctctgccggcccc
accgagcgagcgagcgcgcatagagggagtggccaactccatcactaggggtaacc
Novel AAVrh.39 3′ ITR (SEQ ID NO: 3)
ggttacccctagtgatggagttggccactccctctatgcgcgctcgctcgctcggtggggccggctgagcagagctcagccgtctgcgga
cctttggtccgcaggccccaccgagcgagcgagcgcgcatagagggagtggccaa
Novel AAVrh.39 5′ ITR (SEQ ID NO: 4)
ttggccactccctctatgcgcgctcgctcgctcggtggggcctgcggaccaaaggtccgcagacggctgagctctgctcagccggcccc
accgagcgagcgagcgcgcatagagggagtggccaactccatcactaggggtaacc
Novel AAV8 genome (SEQ ID NO: 5)
ttggccactccctctatgcgcgctcgctcgctcggtggggcctgcggaccaaaggtccgcagacggcagagctctgctctgccggcccc
accgagcgagcgagcgcgcatagagggagtggccaactccatcactaggggtaacctcccacgctgccgcgtcagcgctgacgtaaat
tacgtcataggggagtggtcctgtattagctgtcacgtgagtgcttttgcggcattttgcgacaccacgtggccatttgaggtatatatggccg
agtgagcgagcaggatctccattttgaccgcgaaatttgaacgagcagcagccatgccgggcttctacgagatcgtgatcaaggtgccga
gcgacctggacgagcacctgccgggcatttctgactcgtttgtgaactgggtggccgagaaggaatgggagctgcccccggattctgaca
tggatcggaatctgatcgagcaggcacccctgaccgtggccgagaagctgcagcgcgacttcctggtccaatggcgccgcgtgagtaag
gccccggaggccctcttctttgttcagttcgagaagggcgagagctactttcacctgcacgttctggtcgagaccacgggggtcaagtccat
ggtgctaggccgcttcctgagtcagattcgggagaagctggtccagaccatctaccgcggggtcgagcccacgctgcccaactggttcgc
ggtgaccaagacgcgtaatggcgccggcggggggaacaaggtggtggacgagtgctacatccccaactacctcctgcccaagactcag
cccgagctgcagtgggcgtggactaacatggaggagtatataagcgcgtgcttgaacctggccgagcgcaaacggctcgtggcgcagc
acctgacccacgtcagccagacgcaggagcagaacaaggagaatctgaaccccaattctgacgcgcccgtgatcaggtcaaaaacctcc
gcgcgctatatggagctggtcgggtggctggtggaccggggcatcacctccgagaagcagtggatccaggaggaccaggcctcgtaca
tctccttcaacgccgcctccaactcgcggtcccagatcaaggccgcgctggacaatgccggcaagatcatggcgctgaccaaatccgcg
cccgactacctggtggggccctcgctgcccgcggacattacccagaaccgcatctaccgcatcctcgctctcaacggctacgaccctgcc
tacgccggctccgtctttctcggctgggctcagaaaaagttcgggaaacgcaacaccatctggctgtttggacccgccaccaccggcaag
accaacattgcggaagccatcgcccacgccgtgcccttctacggctgcgtcaactggaccaatgagaactttcccttcaatgattgcgtcga
caagatggtgatctggtgggaggagggcaagatgacggccaaggtcgtggagtccgccaaggccattctcggcggcagcaaggtgcg
cgtggaccaaaagtgcaagtcgtccgcccagatcgaccccacccccgtgatcgtcacctccaacaccaacatgtgcgccgtgattgacgg
gaacagcaccaccttcgagcaccagcagcctctccaggaccggatgtttaagttcgaactcacccgccgtctggagcacgactttggcaa
ggtgacaaagcaggaagtcaaagagttcttccgctgggccagtgatcacgtgaccgaggtggcgcatgagttttacgtcagaaagggcg
gagccagcaaaagacccgcccccgatgacgcggataaaagcgagcccaagcgggcctgcccctcagtcgcggatccatcgacgtcag
acgcggaaggagctccggtggactttgccgacaggtaccaaaacaaatgttctcgtcacgcgggcatgcttcagatgctgtttccctgcaa
aacgtgcgagagaatgaatcagaatttcaacatttgcttcacacacggggtcagagactgctcagagtgtttccccggcgtgtcagaatctc
aaccggtcgtcagaaagaggacgtatcggaaactctgtgcgattcatcatctgctggggcgggctcccgagattgcttgctcggcctgcga
tctggtcaacgtggacctggatgactgtgtttctgagcaataaatgacttaaaccaggtatggctgccgatggttatcttccagattggctcg
aggacaacctctctgagggcattcgcgagtggtgggcgctgaaacctggagccccgaagcccaaagccaaccagcaaaagcaggacga
cggccggggtctggtgcttcctggctacaagtacctcggacccttcaacggactcgacaagggggagcccgtcaacgcggcggacgca
gcggccctcgagcacgacaaggcctacgaccagcagctgcaggcgggtgacaatccgtacctgcggtataaccacgccgacgccgag
tttcaggagcgtctgcaagaagatacgtcttttgggggcaacctcgggcgagcagtcttccaggccaagaagcgggttctcgaacctctcg
gtctggttgaggaaggcgctaagacggctcctggaaagaagagaccggtagagccatcaccccagcgttctccagactcctctacgggc
atcggcaagaaaggccaacagcccgccagaaaaagactcaattttggtcagactggcgactcagagtcagttccagaccctcaacctctc
ggagaacctccagcagcgccctctggtgtgggacctaatacaatggctgcaggcggtggcgcaccaatggcagacaataacgaaggcg
ccgacggagtgggtagttcctcgggaaattggcattgcgattccacatggctgggcgacagagtcatcaccaccagcacccgaacctgg
gccctgcccacctacaacaaccacctctacaagcaaatctccaacgggacatcgggaggagccaccaacgacaacacctacttcggcta
cagcaccccctgggggtattttgactttaacagattccactgccacttttcaccacgtgactggcagcgactcatcaacaacaactggggatt
ccggcccaagagactcagcttcaagctcttcaacatccaggtcaaggaggtcacgcagaatgaaggcaccaagaccatcgccaataacc
tcaccagcaccatccaggtgtttacggactcggagtaccagctgccgtacgttctcggctctgcccaccagggctgcctgcctccgttcccg
gcggacgtgttcatgattccccagtacggctacctaacactcaacaacggtagtcaggccgtgggacgctcctccttctactgcctggaata
ctttccttcgcagatgctgagaaccggcaacaacttccagtttacttacaccttcgaggacgtgcctttccacagcagctacgcccacagcca
gagcttggaccggctgatgaatcctctgattgaccagtacctgtactacttgtctcggactcaaacaacaggaggcacggcaaatacgcag
actctgggcttcagccaaggtgggcctaatacaatggccaatcaggcaaagaactggctgccaggaccctgttaccgccaacaacgcgtc
tcaacgacaaccgggcaaaacaacaatagcaactttgcctggactgctgggaccaaataccatctgaatggaagaaattcattggctaatc
ctggcatcgctatggcaacacacaaagacgacaaggagcgtttttttcccagtaacgggatcctgatttttggcaaacaaaatgctgccaga
gacaatgcggattacagcgatgtcatgctcaccagcgaggaagaaatcaaaaccactaaccctgtggctacagaggaatacggtatcgtg
gcagataacttgcagcagcaaaacacggctcctcaaattggaactgtcaacagccagggggccttacccggtatggtctggcagaaccg
ggacgtgtacctgcagggtcccatctgggccaagattcctcacacggacggcaacttccacccgtctccgctgatgggcggctttggcctg
aaacatcctccgcctcagatcctgatcaagaacacgcctgtacctgcggatcctccgaccaccttcaaccagtcaaagctgaactctttcatc
acgcaatacagcaccggacaggtcagcgtggaaattgaatgggagctgcagaaggaaaacagcaagcgctggaaccccgagatccag
tacacctccaactactacaaatctacaagtgtggactttgctgttaatacagaaggcgtgtactctgaaccccgccccattggcacccgttacc
tcacccgtaatctgtaattgcctgttaatcaataaaccggttgattcgtttcagttgaactttggcctactgtccttcttatcttatctcgtct
ccatggcaactggttaaacattaactGCTTGGGTGCGCTTCGCGATAAGGGACTGACGTCATCgggttacccctag
tgatggagttggccactccctctatgcgcgctcgctcgctcggtggggccggcagagcagagctctgccgtctgcggacctttggtccgca
ggccccaccgagcgagcgagcgcgcatagagggagtggccaa
Novel AAVrh.39 genome (SEQ ID NO: 6)
ttggccactccctctatgcgcgctcgctcgctcggtggggcctgcggaccaaaggtccgcagacggctgagctctgctcagccggcccc
accgagcgagcgagcgcgcatagagggagtggccaactccatcactaggggtaaccGCGAAGCGCCTCCCACGCTG
CCGCGTcagcgctgacgtaaatcacgtcataggggagtggtcctgtattagctgtcacgtgagtgcttttgcgacagtttgcgacacca
cgtggtcacagggggtatatatggccgagtgagcacgcaggatctccattttgagcgcggaatttgaacgagcagcagccatgccgggct
tctacgagatcgtgatcaaggtgccgagcgacctggacgagcacctgccgggcatttctgactcgttcgtgaactgggtggccgagaagg
aatgggagctgcccccggattctgacatggatcggaatctgatcgagcaggcacccctgaccgtggccgagaagctgcagcgcgacttc
ctggtcgaatggcgccgcgtgagtaaggccccggaggccctcttctttgttcagttcgagaagggggaaagctactttcacctgcacgttct
ggtcgagaccacgggggtcaagtccatggtgctgggccgcttcctgagccagattcgcgaaaagctcgtgcaacgcatctaccgcgggg
tcgagcccacgctgcccaactggttcgcggtgaccaagacgcgaaatggcgccggggggggaacaaggtggtggacgagtgctaca
tccccaactacctcctgcccaagacgcagcccgagctgcagtgggcgtggactaacatggaggagtatataagcgcgtgtctgaacctcg
cggagcgtaaacggctcgtggcgcagcacctgacccacgtcagccagacgcaggagcagaacaaggagaatctgaacccgaattctg
acgcgcccgtgatcaggtcaaaaacctccgcgcgctacatggagctggtcgggtggctggtggaccggggcatcacctccgagaagca
gtggatccaggaggaccaggcctcgtacatctccttcaacgccgcctccaactcgcggtcccagatcaaggccgcgctggacaatgccg
gaaagatcatggcgctgaccaaatccgcgcccgactacctggtgggcccgtccttacccgcggacattaaggccaaccgcatctaccgc
atcctggagctcaacggctacgaccccgcctacgccggctccgtcttcctgggctgggcgcagaaaaagttcggtaaacgcaacaccatc
tggctcttcgggcccgccaccaccggcaagaccaacatcgcggaagccatcgcccacgccgtgcccttctacggctgcgtcaactggac
caatgagaactttcccttcaacgattgcgtcgacaagatggtgatctggtgggaggagggcaagatgaccgccaaggtcgtggagtccgc
caaggccattctgggtggaagcaaggtgcgcgtggaccaaaagtgcaagtcatcggcccagatcgaccccacgcccgtgatcgtcacct
ccaacaccaacatgtgcgccgtgatcgacgggaacagcaccaccttcgagcaccagcagcccctgcaggaccgcatgttcaagttcgag
ctcacccgccgtctggagcacgactttggcaaggtgaccaagcaggaagtcaaagagttcttccgctgggctcaggatcacgtgactgag
gtggcgcatgagttctacgtcagaaagggcggagccaccaaaagacccgcccccagtgacgcggatataagcgagcccaagcgggcc
tgcccctcagttgcggagccatcgacgtcagacgcggaagcaccggtggactttgcggacaggtaccaaaacaaatgttctcgtcacgcg
ggcatgcttcagatgctgtttccctgcaagacatgcgagagaatgaatcagaatttcaacgtctgcttcacgcacggggtcagagactgctc
agagtgcttccccggcgcgtcagaatctcaacccgtcgtcagaaaaaagacgtatcagaaactgtgcgcgattcatcatctgctggggcgg
gcacccgagattgcgtgttcggcctgcgatctcgtcaacgtggacttggatgactgtgtttctgagcaataaatgacttaaaccaggtatggc
tgctgacggttatcttccagattggctcgaggacaacctctctgagggcattcgcgagtggtgggacctgaaacctggagcccccaagccc
aaggccaaccagcagaagcaggacgacggccggggtctggtgcttcctggctacaagtacctcggacccttcaacggactcgacaagg
gggagcccgtcaacgcggcggacgcagcggccctcgagcacgacaaggcctacgaccagcagctcaaagcgggtgacaatccgtac
ctgcggtataaccacgccgacgccgagtttcaggagcgtctgcaagaagatacgtcttttgggggcaacctcgggcgagcagtcttccag
gccaagaagcgggttctcgaacctctcggtctggttgaggaagctgctaagacggctcctggaaagaagagaccggtagaaccgtcacc
tcagcgttcccccgactcctccacgggcatcggcaagaaaggccagcagcccgctaaaaagagactgaactttggtcagactggcgact
cagagtcagtccccgaccctcaaccaatcggagaaccaccagcaggcccctctggtctgggatctggtacaatggctgcaggcggtggc
gctccaatggcagacaataacgaaggcgccgacggagtgggtagttcctcaggaaattggcattgcgattccacatggctgggcgacag
agtcatcaccaccagcacccgaacctgggccctgcccacctacaacaaccacctctacaagcaaatatccAATGGGACATCG
GGAGGAAGCACCAACGACAACACCTACTTCGGCTACAGCACCCCCTGGGGGTATTT
TGACTTCAACAGATTCCACTGCCACTTCTCACCACGTGACTGGCAGCGACTCATCAA
CAACAACTGGGGATTCCGGCCAAAAAGACTCAGCTTCAAGCTCTTCAACATCCAGG
TCAAGGAGGTCACGCAGAATGAAGGCACCAAGACCATCGCCAATAACCTTACCAGC
ACGATTCAGGTATTTACGGACTCGGAATACCAGCTGCCGTACGTCCTCGGCTCCGCG
CACCAGGGCTGCCTGCCTCCGTTCCCGGCGGACGTCTTCATGattccccagtacggctaccttacac
tgaacaatggaagtcaagccgtaggccgttcctccttctactgcctggaatattttccatctcaaatgctgcgaactggaaacaattttgaat
tcagctacaccttcgaggacgtgcctttccacagcagctacgcacacagccagagcttggaccgactgatgaatcctctcatcgaccagtacc
tgtactacttatccagaactcagtccacaggaggaactcaaggtacccagcaattgttattttctcaagctgggcctgcaaacatgtcggctc
aggctaagaactggctacctggaccttgctaccggcagcagcgagtctctacgacactgtcgcaaaacaacaacagcaactttgcttggact
ggtgccaccaaatatcacctgaacggaagagactctttggtaaatcccggtgtcgccatggcaacccacaaggacgacgaggaacgcttc
ttcccgtcgagtggagtcctgatgtttggaaaacagggtgctggaagagacaatgtggactacagcagcgttatgctaaccagcgaagaa
gaaattaaaaccactaaccctgtagccacagaacaatacggtgtggtggctgacaacttgcagcaaaccaatacagggcctattgtgggaa
atgtcaacagccaaggagccttacctggcatggtctggcagaaccgagacgtgtacctgcagggtcccatctgggccaagattcctcaca
cggacggcaacttccacccttcaccgctaatgggaggatttggactgaagcacccacctcctcagatcctgatcaagaacacgccggtac
ctgcggatcctccaacaacgttcagccaggcgaaattggcttccttcattacgcagtacagcaccggacaggtcagcgtggaaatcgagtg
ggagctgcagaaggagaacagcaaacgctggaacccagagattcagtacacttcaaactactacaaatctacaaatgtggactttgctgtc
aatacagagggaacttattctgagcctcgccccattggtactcgttacctcacccgtaatctgtaattgctggttaatcaataaaccgtttga
ttcgtttcagttgaactttggtctccgtgtgcttcttatcttatctcgtctccatggcaactggttacacattaactgcttggtgcgcttcgc
GATCATAAATGACTTACGTCATCGggttacccctagtgatggagttggccactccctctatgcgcgctcgctcgctcggtggggc
cggctgagcagagctcagccgtctgcggacctttggtccgcaggccccaccgagcgagcgagcgcgcatagagggagtggccaa
Novel AAV8 Rep78 protein (SEQ ID NO: 7)
MPGFYEIVIKVPSDLDEHLPGISDSFVNWVAEKEWELPPDSDMDRNLIEQAPLTVAEKL
QRDFLVQWRRVSKAPEALFFVQFEKGESYFHLHVLVETTGVKSMVLGRFLSQIREKLV
QTIYRGVEPTLPNWFAVTKTRNGAGGGNKVVDECYIPNYLLPKTQPELQWAWTNMEE
YISACLNLAERKRLVAQHLTHVSQTQEQNKENLNPNSDAPVIRSKTSARYMELVGWLV
DRGITSEKQWIQEDQASYISFNAASNSRSQIKAALDNAGKIMALTKSAPDYLVGPSLPAD
ITQNRIYRILALNGYDPAYAGSVFLGWAQKKFGKRNTIWLFGPATTGKTNIAEAIAHAV
PFYGCVNWTNENFPFNDCVDKMVIWWEEGKMTAKVVESAKAILGGSKVRVDQKCKS
SAQIDPTPVIVTSNTNMCAVIDGNSTTFEHQQPLQDRMFKFELTRRLEHDFGKVTKQEV
KEFFRWASDHVTEVAHEFYVRKGGASKRPAPDDADKSEPKRACPSVADPSTSDAEGAP
VDFADRYQNKCSRHAGMLQMLFPCKTCERMNQNFNICFTHGVRDCSECFPGVSESQPV
VRKRTYRKLCAIHHLLGRAPEIACSACDLVNVDLDDCVSEQ*
Novel AAV8 Rep68 protein (SEQ ID NO: 8)
MPGFYEIVIKVPSDLDEHLPGISDSFVNWVAEKEWELPPDSDMDRNLIEQAPLTVAEKL
QRDFLVQWRRVSKAPEALFFVQFEKGESYFHLHVLVETTGVKSMVLGRFLSQIREKLV
QTIYRGVEPTLPNWFAVTKTRNGAGGGNKVVDECYIPNYLLPKTQPELQWAWTNMEE
YISACLNLAERKRLVAQHLTHVSQTQEQNKENLNPNSDAPVIRSKTSARYMELVGWLV
DRGITSEKQWIQEDQASYISFNAASNSRSQIKAALDNAGKIMALTKSAPDYLVGPSLPAD
ITQNRIYRILALNGYDPAYAGSVFLGWAQKKFGKRNTIWLFGPATTGKTNIAEAIAHAV
PFYGCVNWTNENFPFNDCVDKMVIWWEEGKMTAKVVESAKAILGGSKVRVDQKCKS
SAQIDPTPVIVTSNTNMCAVIDGNSTTFEHQQPLQDRMFKFELTRRLEHDFGKVTKQEV
KEFFRWASDHVTEVAHEFYVRKGGASKRPAPDDADKSEPKRACPSVADPSTSDAEGAP
VDFADRLARGHSL*
Novel AAV8 Rep52 protein (SEQ ID NO: 9)
MELVGWLVDRGITSEKQWIQEDQASYISFNAASNSRSQIKAALDNAGKIMALTKSAPD
YLVGPSLPADITQNRIYRILALNGYDPAYAGSVFLGWAQKKFGKRNTIWLFGPATTGKT
NIAEAIAHAVPFYGCVNWTNENFPFNDCVDKMVIWWEEGKMTAKVVESAKAILGGSK
VRVDQKCKSSAQIDPTPVIVTSNTNMCAVIDGNSTTFEHQQPLQDRMFKFELTRRLEHD
FGKVTKQEVKEFFRWASDHVTEVAHEFYVRKGGASKRPAPDDADKSEPKRACPSVAD
PSTSDAEGAPVDFADRYQNKCSRHAGMLQMLFPCKTCERMNQNFNICFTHGVRDCSEC
FPGVSESQPVVRKRTYRKLCAIHHLLGRAPEIACSACDLVNVDLDDCVSEQ*
Novel AAV8 Rep40 protein (SEQ ID NO: 10)
MELVGWLVDRGITSEKQWIQEDQASYISFNAASNSRSQIKAALDNAGKIMALTKSAPD
YLVGPSLPADITQNRIYRILALNGYDPAYAGSVFLGWAQKKFGKRNTIWLFGPATTGKT
NIAEAIAHAVPFYGCVNWTNENFPFNDCVDKMVIWWEEGKMTAKVVESAKAILGGSK
VRVDQKCKSSAQIDPTPVIVTSNTNMCAVIDGNSTTFEHQQPLQDRMFKFELTRRLEHD
FGKVTKQEVKEFFRWASDHVTEVAHEFYVRKGGASKRPAPDDADKSEPKRACPSVAD
PSTSDAEGAPVDFADRLARGHSL*
Novel AAVrh.39 Rep78 protein (SEQ ID NO: 11)
MPGFYEIVIKVPSDLDEHLPGISDSFVNWVAEKEWELPPDSDMDRNLIEQAPLTVAEKL
QRDFLVEWRRVSKAPEALFFVQFEKGESYFHLHVLVETTGVKSMVLGRFLSQIREKLV
QRIYRGVEPTLPNWFAVTKTRNGAGGGNKVVDECYIPNYLLPKTQPELQWAWTNMEE
YISACLNLAERKRLVAQHLTHVSQTQEQNKENLNPNSDAPVIRSKTSARYMELVGWLV
DRGITSEKQWIQEDQASYISFNAASNSRSQIKAALDNAGKIMALTKSAPDYLVGPSLPAD
IKANRIYRILELNGYDPAYAGSVFLGWAQKKFGKRNTIWLFGPATTGKTNIAEAIAHAV
PFYGCVNWTNENFPFNDCVDKMVIWWEEGKMTAKVVESAKAILGGSKVRVDQKCKS
SAQIDPTPVIVTSNTNMCAVIDGNSTTFEHQQPLQDRMFKFELTRRLEHDFGKVTKQEV
KEFFRWAQDHVTEVAHEFYVRKGGATKRPAPSDADISEPKRACPSVAEPSTSDAEAPV
DFADRYQNKCSRHAGMLQMLFPCKTCERMNQNFNVCFTHGVRDCSECFPGASESQPV
VRKKTYQKLCAIHHLLGRAPEIACSACDLVNVDLDDCVSEQ*
Novel AAVrh.39 Rep68 protein (SEQ ID NO: 12)
MPGFYEIVIKVPSDLDEHLPGISDSFVNWVAEKEWELPPDSDMDRNLIEQAPLTVAEKL
QRDFLVEWRRVSKAPEALFFVQFEKGESYFHLHVLVETTGVKSMVLGRFLSQIREKLV
QRIYRGVEPTLPNWFAVTKTRNGAGGGNKVVDECYIPNYLLPKTQPELQWAWTNMEE
YISACLNLAERKRLVAQHLTHVSQTQEQNKENLNPNSDAPVIRSKTSARYMELVGWLV
DRGITSEKQWIQEDQASYISFNAASNSRSQIKAALDNAGKIMALTKSAPDYLVGPSLPAD
IKANRIYRILELNGYDPAYAGSVFLGWAQKKFGKRNTIWLFGPATTGKTNIAEAIAHAV
PFYGCVNWTNENFPFNDCVDKMVIWWEEGKMTAKVVESAKAILGGSKVRVDQKCKS
SAQIDPTPVIVTSNTNMCAVIDGNSTTFEHQQPLQDRMFKFELTRRLEHDFGKVTKQEV
KEFFRWAQDHVTEVAHEFYVRKGGATKRPAPSDADISEPKRACPSVAEPSTSDAEAPV
DFADRLARGHSL*
Novel AAVrh.39 Rep52 protein (SEQ ID NO: 13)
MELVGWLVDRGITSEKQWIQEDQASYISFNAASNSRSQIKAALDNAGKIMALTKSAPD
YLVGPSLPADIKANRIYRILELNGYDPAYAGSVFLGWAQKKFGKRNTIWLFGPATTGKT
NIAEAIAHAVPFYGCVNWTNENFPFNDCVDKMVIWWEEGKMTAKVVESAKAILGGSK
VRVDQKCKSSAQIDPTPVIVTSNTNMCAVIDGNSTTFEHQQPLQDRMFKFELTRRLEHD
FGKVTKQEVKEFFRWAQDHVTEVAHEFYVRKGGATKRPAPSDADISEPKRACPSVAEP
STSDAEAPVDFADRYQNKCSRHAGMLQMLFPCKTCERMNQNFNVCFTHGVRDCSECF
PGASESQPVVRKKTYQKLCAIHHLLGRAPEIACSACDLVNVDLDDCVSEQ*
Novel AAVrh.39 Rep40 protein (SEQ ID NO: 14)
MELVGWLVDRGITSEKQWIQEDQASYISFNAASNSRSQIKAALDNAGKIMALTKSAPD
YLVGPSLPADIKANRIYRILELNGYDPAYAGSVFLGWAQKKFGKRNTIWLFGPATTGKT
NIAEAIAHAVPFYGCVNWTNENFPFNDCVDKMVIWWEEGKMTAKVVESAKAILGGSK
VRVDQKCKSSAQIDPTPVIVTSNTNMCAVIDGNSTTFEHQQPLQDRMFKFELTRRLEHD
FGKVTKQEVKEFFRWAQDHVTEVAHEFYVRKGGATKRPAPSDADISEPKRACPSVAEP
STSDAEAPVDFADRLARGHSL*
Novel AAV8 cap protein (SEQ ID NO: 15)
MAADGYLPDWLEDNLSEGIREWWALKPGAPKPKANQQKQDDGRGLVLPGYKYLGPF
NGLDKGEPVNAADAAALEHDKAYDQQLQAGDNPYLRYNHADAEFQERLQEDTSFGG
NLGRAVFQAKKRVLEPLGLVEEGAKTAPGKKRPVEPSPQRSPDSSTGIGKKGQQPARKR
LNFGQTGDSESVPDPQPLGEPPAAPSGVGPNTMAAGGGAPMADNNEGADGVGSSSGN
WHCDSTWLGDRVITTSTRTWALPTYNNHLYKQISNGTSGGATNDNTYFGYSTPWGYF
DFNRFHCHFSPRDWQRLINNNWGFRPKRLSFKLFNIQVKEVTQNEGTKTIANNLTSTIQ
VFTDSEYQLPYVLGSAHQGCLPPFPADVFMIPQYGYLTLNNGSQAVGRSSFYCLEYFPS
QMLRTGNNFQFTYTFEDVPFHSSYAHSQSLDRLMNPLIDQYLYYLSRTQTTGGTANTQT
LGFSQGGPNTMANQAKNWLPGPCYRQQRVSTTTGQNNNSNFAWTAGTKYHLNGRNS
LANPGIAMATHKDDKERFFPSNGILIFGKQNAARDNADYSDVMLTSEEEIKTTNPVATE
EYGIVADNLQQQNTAPQIGTVNSQGALPGMVWQNRDVYLQGPIWAKIPHTDGNFHPSP
LMGGFGLKHPPPQILIKNTPVPADPPTTFNQSKLNSFITQYSTGQVSVEIEWELQKENSKR
WNPEIQYTSNYYKSTSVDFAVNTEGVYSEPRPIGTRYLTRNL*
Novel AAVrh.39 cap protein (SEQ ID NO: 16)
MAADGYLPDWLEDNLSEGIREWWDLKPGAPKPKANQQKQDDGRGLVLPGYKYLGPF
NGLDKGEPVNAADAAALEHDKAYDQQLKAGDNPYLRYNHADAEFQERLQEDTSFGG
NLGRAVFQAKKRVLEPLGLVEEAAKTAPGKKRPVEPSPQRSPDSSTGIGKKGQQPAKK
RLNFGQTGDSESVPDPQPIGEPPAGPSGLGSGTMAAGGGAPMADNNEGADGVGSSSGN
WHCDSTWLGDRVITTSTRTWALPTYNNHLYKQISNGTSGGSTNDNTYFGYSTPWGYFD
FNRFHCHFSPRDWQRLINNNWGFRPKRLSFKLFNIQVKEVTQNEGTKTIANNLTSTIQVF
TDSEYQLPYVLGSAHQGCLPPFPADVFMIPQYGYLTLNNGSQAVGRSSFYCLEYFPSQM
LRTGNNFEFSYTFEDVPFHSSYAHSQSLDRLMNPLIDQYLYYLSRTQSTGGTQGTQQLL
FSQAGPANMSAQAKNWLPGPCYRQQRVSTTLSQNNNSNFAWTGATKYHLNGRDSLV
NPGVAMATHKDDEERFFPSSGVLMFGKQGAGRDNVDYSSVMLTSEEEIKTTNPVATEQ
YGVVADNLQQTNTGPIVGNVNSQGALPGMVWQNRDVYLQGPIWAKIPHTDGNFHPSP
LMGGFGLKHPPPQILIKNTPVPADPPTTFSQAKLASFITQYSTGQVSVEIEWELQKENSKR
WNPEIQYTSNYYKSTNVDFAVNTEGTYSEPRPIGTRYLTRNL*
AAV2 ITR nucleic acid sequence (SEQ ID NO: 17)
ttggccactccctctctgcgcgctcgctcgctcactgaggccgcccgggcaaagcccgggcgtcgggcgacctttggtcgcccggcctc
agtgagcgagcgagcgcgcagagagggagtggccaactccatcactaggggttcct

1.-51. (canceled)

52. A recombinant adeno-associated viral (rAAV) vector comprising a transgene flanked by inverted terminal repeats (ITRs), wherein a first ITR comprises the entirety of the nucleic acid sequence of any one of SEQ ID NOs: 1-4.

53. The rAAV vector of claim 52, wherein the first ITR is an AAV8 ITR and a second ITR is an AAVrh.39 ITR.

54. The rAAV vector of claim 52, wherein the first ITR consists of the nucleic acid sequence of SEQ ID NO: 1.

55. The rAAV vector of claim 52, wherein the first ITR consists of the nucleic acid sequence of SEQ ID NO: 2.

56. The rAAV vector of claim 52, wherein the first ITR consists of the nucleic acid sequence of SEQ ID NO: 3.

57. The rAAV vector of claim 52, wherein the first ITR consists of the nucleic acid sequence of SEQ ID NO: 4.

58. A recombinant adeno-associated virus (rAAV) comprising:
(i) the rAAV vector of claim 52; and
(ii) an AAV capsid protein, optionally wherein the AAV capsid protein comprises the amino acid sequence set forth in SEQ ID NOs: 15 or 16.

59. A host cell comprising the rAAV vector of claim 52.

60. The host cell of claim 59, further comprising a Rep protein having the amino acid sequence set forth in any one of SEQ ID NOs: 7-14 and/or a Cap protein having the amino acid sequence set forth in SEQ ID NO: 15 or 16.

61. A method for delivering a transgene to a cell or subject, the method comprising administering to the cell or subject the rAAV of claim 58.

62. A recombinant adeno-associated viral (rAAV) vector comprising a transgene flanked by inverted terminal repeats (ITRs), wherein a first ITR comprises a nucleic acid sequence having at least 95% identity to the nucleic acid sequence of SEQ ID NOs: 1 or 3.

63. The rAAV vector of claim 62, wherein the first ITR comprises a nucleic acid sequence having at least 95% identity to the nucleic acid sequence of SEQ ID NO: 1.

64. The rAAV vector of claim 62, wherein the first ITR comprises a nucleic acid sequence having at least 95% identity to the nucleic acid sequence of SEQ ID NO: 3.

65. A recombinant adeno-associated virus (rAAV) comprising:
(i) the rAAV vector of claim 62; and
(ii) a AAV capsid protein, optionally wherein the AAV capsid protein comprises the amino acid sequence set forth in SEQ ID NOs: 15 or 16.

66. A host cell comprising the rAAV vector of claim 62.

67. The host cell of claim 66, further comprising a Rep protein having the amino acid sequence set forth in any one of SEQ ID NOs: 7-14 and/or a Cap protein having the amino acid sequence set forth in any one of SEQ ID NO: 15 or 16.

68. A method for delivering a transgene to a cell or subject, the method comprising administering to the cell or subject the rAAV of claim 65.

69. A nucleic acid comprising:
(a) a transgene flanked by inverted terminal repeats (ITRs);
(b) a rep gene encoding an amino acid sequence as set forth in any one of SEQ ID NOS: 7-14; and
(c) a cap gene, optionally wherein the cap gene encodes an amino acid sequence as set forth in SEQ ID NOs: 15 or 16.

70. The nucleic acid of claim 69, wherein a first ITR comprises the entirety of the nucleic acid sequence of any one of SEQ ID NOs: 1-4.

71. The nucleic acid of claim 70, wherein a first ITR is an AAV8 ITR and a second ITR is an AAVrh.39 ITR.
